(19) United States
(12) Patent Application Publication
(10) Pub. No.: US 2009/0176285 A1
(43) Pub. Date: Jul. 9, 2009

(54) Compositions and Methods for Enhancing Glycerol Utilization

(76) Inventors: Genrich Burd, Oak Park, IL (US); Anamitra Bhattacharyya, Evanston, IL (US)

Correspondence Address: POLSINELLI SHUGHART PC, 700 W. 47TH STREET, SUITE 1000, KANSAS CITY, MO, 64112-1802, US

(21) Appl. No.: 12/346,550

(22) Filed: Dec. 30, 2008

Related U.S. Application Data
(63) Continuation-in-part of application No. PCT/US2007/072882, filed on Jul. 5, 2007.
(60) Provisional application No. 60/818,570, filed on Jul. 6, 2006.

(51) Int. Cl. C12P 7/64; C12N 1/19; C12N 1/21; C02F 3/34; C12P 7/52; C12P 7/06; C12P 7/18; C12P 3/00; C12P 7/28

(52) U.S. Cl. 435/134; 435/254.21; 435/252.33; 435/262; 435/141; 435/161; 435/158; 435/168; 435/150

(57) ABSTRACT

A glycerol utilizing cell and a method for the production of glycerol-derived target compounds are provided. The glycerol utilizing cell may comprise a glycerol metabolizing system or a glycerol uptake protein and be used to produce a glycerol-derivable target compound.

### CROSS-RELATED APPLICATIONS

This is a continuation-in-part of PCT/US2007/072882 filed on Jul. 5, 2007, which claims the benefit of U.S. provisional application 60/818,570, filed on Jul. 6, 2006, the contents each of which are incorporated herein by reference.

### FIELD OF THE INVENTION

The present invention relates to methods and compositions for the production of end-product derivatives of glycerol.

### BACKGROUND

Glycerol is formed as a by-product during the production of biodiesel. The availability of crude glycerol is predicted to increase over the next several years as a result of the tremendous growth in biodiesel production. The current surplus of glycerol is already resulting in the shutdown of traditional glycerol-producing plants. In addition, this excess glycerol is causing disposal problems for the oleo-chemical industry, for which glycerol refining represents a long existing revenue source.

Previous attempts to find new applications of glycerol as a low-cost substrate for producing functional end-product derivatives have centered on the use of recombinant, glycerol-metabolizing bacteria. Genes encoding various glycerol metabolic enzymes have been cloned and over-expressed in cells to increase the host strain's production of a valued product. However, these efforts do not address what many consider to be the rate-limiting step of glycerol consumption: glycerol uptake.

There have also been attempts to increase the availability of exogenous glycerol in bacterial fermentation media. However, exogenous glycerol concentrations of 2.0% or more may inhibit cell growth. Furthermore, these efforts do not address the uptake and conversion of glycerol from contaminated sources of glycerol, such as biodiesel production waste. Biodiesel production waste may contain contaminating compounds that inhibit bacterial growth. The waste may be diluted; however, this may result in insufficient glycerol levels for effective production of end-product.

There remains a need to develop a suitable technology for bioproduction of value-added products derived from various sources of glycerol.

### SUMMARY

Provided herein is a microbial cell capable of being used for producing compounds derived from glycerol. The microbial cell may be a glycerol-utilizing cell. The glycerol-utilizing cell may comprise a glycerol metabolizing system and/or a glycerol uptake protein. The cell may express components of the glycerol metabolizing system and the glycerol uptake protein from heterologous nucleic acid or from endogenous nucleic acid. Components of the glycerol metabolizing system may include any of glycerol kinase, glycerol dehydrogenase, glycerol dehydratase, 1,3-propanediol oxidoreductase, dihydroxyacetone (glycerone) kinase, alcohol dehydrogenase, alcohol dehydrogenase (NADP), D-glyceraldehyde dehydrogenase, glycerol-3-phosphate dehydrogenase (NAD(P)), 3-Phospho-D-glycerate dehydrogenase, glycerol-3-phosphate oxidase, glycerol oxidase, glycerol-1-phosphatase, propanediol-phosphate dehydrogenase, aldehyde reductase (dehydrogenase), aldehyde reductase (dehydrogenase) (NAD), glycerol dehydrogenase, 1,3-propanediol dehydrogenase, propanediol dehydratase, 1,3-propanediol dehydratase, and/or glycerate kinase.

Glycerol uptake proteins may be a glycerol facilitator, a glycerol-specific ATP-dependent transporter, and/or a proton/glycerol symporter. Furthermore, the glycerol-utilizing cell may also be resistant to toxicity associated with uptake and metabolism of extracellular glycerol. A cell that is resistant to glycerol-associated toxicity may be the microbial cell deposited in the ATCC (E. coli K12:MG1655-R3/1 as identifier IV638653-39710; ATCC No. PTA-8496).

A method of increasing glycerol utilization in a microbial cell is also provided herein. The glycerol utilizing cell may be contacted with a composition comprising glycerol. The level of glycerol utilization may be compared to a corresponding wild-type cell.

A method of using the glycerol utilizing cells to produce a glycerol-derived target compound is also provided herein. The glycerol-utilizing cell may be contacted with a glycerol composition under suitable conditions for the cell to produce a target compound. The glycerol utilizing cell may be characterized by increased glycerol metabolism. The glycerol-utilizing cell may have increased glycerol metabolism as compared to a corresponding wild-type cell.

The target compounds that may be produced from the method may propionic acid, ethanol, 1,3-propanediol, 1,2-propanediol, 3-hydroxypropionic acid, poly (3-hydroxy-butyrate), poly (3-mercapto-propionate), hydrogen, succinate, dihydroxyacetone, butyric acid, acetic acid, polyglutamic acid, cinnamic acid, rhamnolipids, 3-hydroxacetone, omega-3 polyunsaturated fatty acids, malate, oxaloacetate, fumarate, aconitate, citrate, isocitrate, 2-ketoglutarate, glycerol-3-phosphate, pyruvate, L-lactate, D-lactate, formate. In addition, amino acids, nucleobases, vitamins, antibiotics, and/or propylene glycol may be produced.

A composition comprising the glycerol-utilizing cell and the glycerol composition or substrate is also provided herein.

### BRIEF DESCRIPTION OF THE DRAWINGS

FIG. 1 shows growth of the MG1655 (blue triangles) and MG1655-R3/1 (gly-R; red circles) strains on M9 medium supplemented with glycerol. M9 medium containing different concentrations of glycerol was inoculated with glycerol-preadapted cultures grown overnight in M9 medium containing 0.2% of glycerol. The cultures were diluted 1:100 and incubated with aeration (250 rpm) at 37° C. OD₆₀₀ was measured after 20 hours growth. The results shown are an average of three independent experiments.

FIG. 2 shows growth properties of E. coli (MG1655) wild-type and mutants on solid M9 minimal medium plates containing pure glycerol (A) minimal M9 medium containing 4.5% glycerol (B) minimal M9 medium containing 9% glycerol (C) additional mutants were derived from the mutant strain R3 growing on minimal M9 medium with 7% glycerol. Note that wt refers to wild-type E. coli MG1655; GLR denotes mutant derived from the wt parental strain; R3 refers to additional mutant strains derived from the GLR mutant strain.

FIG. 3 shows that there is no significant difference in the rate of glycerol utilization by wild type strain MG1655 and R3/1 mutant; resistance to glycerol was not the result of a decrease in glycerol utilization.

FIGS. 4A-C shows conserved regions between E. coli GlpF, glycerol facilitator protein and other glycerol facilitator class of proteins across different genera of microorganisms. The genera of microorganisms displayed in FIGS. 4A-C numbered 1-19 are as follows: (1) E. coli protein GlpF (GenBank accession: NP_{—}418362.1); (2) E. coli ol57:H7/glycerol facilitator protein (GenBank Accession No. NP_{—}290556.1); (3) Shigella dysenteriae 1012/glycerol uptake facilitator (GenBank Accession No. ZP_{—}00921256.1); (4) Shigella flexneri/glycerol uptake facilitator protein sp. (GenBank Accession No. P31140); (5) E. coli HS/glycerol uptake facilitator (ZP_{—}00707869.1); (6) Shigella boydii Sb227/facilitated diffusion of glycerol (Gen Bank Accession No. YP_{—}410223.1); (7) E. coli HB101/glycerol diffusion facilitator protein (Gen Bank Accession No. AAA21363.1); (8) Shigella boydii BS512/glycerol uptake facilitator (Gen Bank Accession No. ZP_{—}00696814.1) (9) E. coli K12/unnamed (GenBank Accession CAA33153.1); (10) Salmonella enterica/glycerol uptake facilitator protein (GenBank Accession No. refINP_{—}457965.1); (11) Salmonella typhimurium LT2/glycerol diffusion (GenBank Accession No. refINP_{—}462968.1); (12) Uncultured bacterium/GlpF (GenBank Accession No. gb|AAO59936.1); (13) Enterobacter sp. 638/MIP family channel protein (GenBank Accession No. reflYP_{—}001178752.1); (14) Yersinia mollaretii ATCC 43969/glycerol uptake facilitator (GenBank Accession No. reflZP_{—}00824891.1); (15) glycerol uptake facilitator protein [Yersinia pestis C092] (GenBank Accession No. refINP_{—}403753.1); (16) Glycerol uptake facilitator and related permeases (Major Intrinsic Protein Family) [Yersinia bercovieri ATCC 43970] (GenBank Accession No. reflZP_{—}00821450.1); (17) glycerol uptake facilitator protein [Yersinia enterocolitica subsp. enterocolitica 8081] (GenBank Accession No. reflYP_{—}001004494.1); (18) Glycerol uptake facilitator and related permeases (Major Intrinsic Protein Family) [Yersinia intermedia ATCC 29909] (GenBank Accession No. reflZP_{—}00832843.1); and (19) Glycerol uptake facilitator and related permeases (Major Intrinsic Protein Family) [Yersinia frederiksenii ATCC 33641] (GenBank Accession No. reflZP_{—}00828528.1).

FIG. 5A-D shows conserved regions between Mycoplasma mycoides GtsA (GenBank Accession No. AF251037) and other gtsA glycerol transporter subunit A proteins across different genera of micoorganisms. The other gtsA glycerol transporter subunit A proteins from different genera of microorganisms are displayed in FIGS. 5A-D and numbered 1-8 by their NCBI GenBank Accession No. as follows: ((1) GenBank Accession No. AAG41804.1; (2) GenBank Accession No. NP_{—}975502.1; (3) GenBank Accession No. YP_{—}424-428.1; (4) GenBank Accession No. YP_{—}278506.1; (5) GenBank Accession No. YP_{—}279174.1; (6) GenBank Accession No. YP_{—}287773.1; (7) GenBank Accession No. YP_{—}115902.1; (8) NP_{—}853028.1.

FIG. 6A-C shows conserved regions between Mycoplasma mycoides GtsB (GenBank Accession No. 975503.1) and other gtsB glycerol transporter subunit B proteins across different genera of micoorganisms. The other gtsB glycerol transporter subunit B proteins from different genera of microorganisms are displayed in FIGS. 6A-C and numbered 1-8 by their NCBI GenBank Accession No. as follows: (1) GenBank Accession No. NP_{—}975503.1; (2) GenBank Accession No. CAD12045.1; (3) GenBank Accession No. AAF24205.1; (4) GenBank Accession No. YP_{—}424-427.1; (5) GenBank Accession No. YP_{—}001256374.1; (6) GenBank Accession No. YP_{—}278507.1.

FIG. 7A-C shows conserved regions between Mycoplasma mycoides GtsC (GenBank Accession No. AAG41804.1) and other gtsC glycerol transporter subunit C proteins across different genera of micoorganisms. The other gtsC glycerol transporter subunit C proteins from different genera of microorganisms are displayed in FIGS. 7A-C and numbered 1-8 by their NCBI GenBank Accession No. as follows: (1) GenBank Accession No. AAG41806.1; (2) GenBank Accession No. NP_{—}975504.1; (3) GenBank Accession No. YP_{—}424-426.1; (4) GenBank Accession No. CAD12046.1; (5) GenBank Accession No. YP_{—}001256792.1; (6) GenBank Accession No. YP_{—}278508.1.

FIG. 8A-E shows conserved regions between Yeast STL1, sugar transporter (GenBank Accession No. P39932) and other STL1 sugar transporter like proteins across different genera of micoorganisms. The other STL1 sugar transporter proteins are from different genera of microorganisms and are displayed in FIGS. 8A-E and numbered 1-22 by their NCBI GenBank Accession No. as follows: (1) GenBank Accession No. P39932; (2) GenBank Accession No. AAU09713.1; (3) GenBank Accession No. AAA57229.1; (4) GenBank Accession No. XP_{—}456249.1; (5) GenBank Accession No. XP_{—}456249.1; (6) GenBank Accession No. NP_{—}984235.1; (7) GenBank Accession No. XP_{—}001524136.1; (8) GenBank Accession No. XP_{—}459387.1; (9) GenBank Accession No. XP_{—}718089.1; (10) GenBank Accession No. XP_{—}001483277.1; (11) GenBank Accession No. XP_{—}001383774.1: (12) GenBank Accession No. XP_{—}001484307.1; (13) GenBank Accession No. XP_{—}001524137.1; (14) GenBank Accession No. XP_{—}457182.1; (15) GenBank Accession No. XP_{—}459386.1; (16) GenBank Accession No. XP_{—}460384.1; (17) GenBank Accession No. XP_{—}001209239.1; (18) GenBank Accession No. BAE63839.1; (19) GenBank Accession No. XP_{—}682437.1; (20) GenBank Accession No. XP_{—}747372.1; (21) GenBank Accession No. XP_{—}001262116.1; (21) GenBank Accession No. XP_{—}001216538.1

### DETAILED DESCRIPTION

Current methods for utilizing glycerol as a low-cost substrate for the production of end-product derivatives from contaminated sources may be cost prohibitive and inefficient. An impediment in the field of biodiesel production is finding a profitable use for the glycerol-containing by-product of biodiesel manufacture reactions.

Provided herein are glycerol utilizing cells capable of being used in methods to bioproduce valued products from glycerol compositions. Glycerol transporters may be cloned into cells capable of metabolizing increased levels of glycerol influx. This may allow for efficient production of end-product functional derivatives of glycerol. The glycerol utilizing cells may have increased glycerol metabolizing capabilities as compared to their wild-type counterparts. The glycerol compositions may have increased concentrations of glycerol and/or glycerol sources contaminated with potential cell-growth inhibiting compounds. The cells may be resistant to toxicity associated with increased glycerol utilization. The cells may be mixed or combined with a solution or substrate comprising glycerol to form a composition. The glycerol may be crude glycerol. The cell-glycerol solution or substrate may be used to produce glycerol-derived products.

### 1. DEFINITIONS

The terminology used herein is for the purpose of describing particular embodiments only and is not intended to be limiting. As used in the specification and the appended claims, the singular forms “a,” “an” and “the” include plural referents unless the context clearly dictates otherwise.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

a. “Cloning Site”

“Cloning site” as used herein may mean a region that allows for the insertion of desired nucleic acid sequences. Typically, the cloning site comprises one or more restriction endonuclease recognition sites. Cloning sites may include multiple cloning sites or polylinkers.

b. “Expression”

“Expression” as used herein may mean the transcription and translation to gene product from a gene coding for the sequence of the gene product.

c. “Gene”

“Gene” as used herein may mean a nucleic acid that expresses a specific protein, including regulatory sequences preceding (5′ non-coding) and following (3′ non-coding) the coding region. The terms “native” and “wild-type” refer to a gene as found in nature with its own regulatory sequences.

d. “Glycerol-Associated Toxicity”

“Glycerol-associated toxicity” may mean a bacteriostatic effect that is associated with a decrease in bacterial growth rate in the presence of glycerol in the growth medium. Glycerol concentrations of 2% or greater in the growth medium may cause the bacteriostatic effect that is associated with a decrease in bacterial growth rate.

e. “Heterologous”

“Heterologous,” “foreign gene,” “foreign nucleic acid,” and “heterologous gene” as used herein may mean a genetic material native to one organism that has been placed within a host organism by various means. The gene of interest may be a naturally occurring gene, a mutated gene or a synthetic gene.

f. “Homologous”

“Homologous” as used herein may mean a high degree of sequence identity between two polypeptides, or a high degree of similarity between the three-dimensional structure or to a high degree of similarity between the active site and the mechanism of action.

g. “Isolated”

“Isolated” as used herein may mean a protein or nucleic acid sequence that is removed from at least one component with which it is naturally associated.

h. Nucleic Acid Fragment

“Nucleic acid fragment” as used herein may mean a nucleic acid that may be employed at any length, with the total length being limited by the ease of preparation and use in the intended recombinant nucleic acid protocol. Illustrative nucleic acid segments may be useful with total lengths of about 10,000, about 5000, about 3000, about 2,000, about 1,000, about 500, about 200, about 100, about 50 base pairs in length, and the like.

i. “Origin of Replication”

“Origin of replication” as used herein may mean a nucleic acid sequence that is necessary allow replication of a plasmid within an organism.

j. “Promoter”

“Promoter” as used herein may mean a nucleic acid fragment to which ribonucleic acid polymerase binds to initiate the transcription of nucleic acid sequences linked to the promoter.

k. “Recombinant Organism”

“Recombinant organism” and “transformed host” as used herein may mean any organism having been transformed with heterologous or foreign genes or extra copies of homologous genes.

l. Substantially Complementary

“Substantially complementary” as used herein may mean that a first sequence is at least 50%, 55%, 60%, 65%, 70%, 75%, 80%, 85%, 90%, 95%, 96%, 97%, 98%, or 99% identical to the complement of a second sequence over a a region of 8, 9, 10, 20, 30, 40, 50, 60, 70, 80, 90, 100, 150, 200, 250, 300, 350 or more nucleotides or amino acids nucleotides, or amino acids. Intermediate lengths may mean any length between the quoted values, such as 16, 17, 18, 19, etc.; 21, 22, 23, etc.; 30, 31, 32, etc.; 50, 51, 52, 53, etc.; 100, 101, 102, 103, etc.; 150, 151, 152, 153, etc.; including all integers through 200-500; 500-1,000, and the like. Substantial complementary may also mean that the two nucleotide sequences hybridize under stringent hybridization conditions.

m. Substantially Identical

“Substantially identical” as used herein may mean that a first and second nucleotide or amino acid sequence are at least about 60%, 65%, 70%, 75%, 80%, 85%, 90%, 91%, 92%, 93%, 94%, 95%, 96%, 97%, 98%, or 99% identical over a region of 8, 9, 10, 20, 30, 40, 50, 60, 70, 80, 90, 100, 150, 200, 250, 300, 350 or more nucleotides or amino acids. Intermediate lengths may mean any length between the quoted values, such as 16, 17, 18, 19, etc.; 21, 22, 23, etc.; 30, 31, 32, etc.; 50, 51, 52, 53, etc.; 100, 101, 102, 103, etc.; 150, 151, 152, 153, etc.; including all integers through 200-500; 500-1,000, and the like. Substantially identical may also mean the first sequence nucleotide or amino acid sequence is substantially complementary to the complement of the second sequence.

n. “Transformation”

“Transformation” as used herein may mean the process of introducing nucleic acid into an organism which changes the genotype of the recipient organism (i.e. the acquisition of new genes in a cell after the incorporation of nucleic acid. The acquired genes may be integrated into chromosomal DNA or introduced as extrachromosomal replicating sequences.). The term “transformant” refers to the product of a transformation.

o. Variant

“Variant” as used herein in the context of a nucleic acid may mean a substantially identical or substantially complementary sequence. A variant in reference to a nucleic acid may further mean a nucleic acid that may contain one or more substitutions, additions, deletions, insertions, or may be fragments thereof. A variant may also be a nucleic acid capable of hybridizing under moderately stringent conditions and specifically binding to a nucleic acid encoding the agent.

A variant in reference to a peptide may further mean differing from a native peptide in one or more substitutions, deletions, additions and/or insertions, or a sequence substantially identical to the native peptide sequence. The ability of a variant to react with antigen-specific antisera may be enhanced or unchanged, relative to the native protein, or may be diminished by less than 50%, or less than 20%, relative to the native peptide. Such variants may generally be identified by modifying one of the peptide sequences encoding an agent and evaluating the reactivity of the modified peptide with antigen-specific antibodies or antisera as described herein. Variants may include those in which one or more portions have been removed such as an N-terminal leader sequence or transmembrane domain. Other variants may include variants in which a small portion (e.g., 1-30 amino acids, or 5-15 amino acids) has been removed from the N- and/or C-terminal of the mature protein.

A variant in reference to a peptide may contain conservative substitutions. A “conservative substitution” is one in which an amino acid is substituted for another amino acid that has similar properties, such that one skilled in the art of peptide chemistry would expect the secondary structure and hydropathic nature of the polypeptide to be substantially unchanged. Amino acid substitutions may generally be made on the basis of similarity in polarity, charge, solubility, hydrophobicity, hydrophilicity and/or the amphipathic nature of the residues. For example, negatively charged amino acids include aspartic acid and glutamic acid; positively charged amino acids include lysine and arginine; and amino acids with uncharged polar head groups having similar hydrophilicity values include leucine, isoleucine and valine; glycine and alanine; asparagine and glutamine; and serine, threonine, phenylalanine and tyrosine. Other groups of amino acids that may represent conservative changes include: (1) ala, pro, gly, glu, asp, gin, asn, ser, thr; (2) cys, ser, tyr, thr; (3) val, ile, leu, met, ala, phe; (4) lys, arg, his; and (5) phe, tyr, trp, his. A variant may also contain nonconservative changes. Variant peptides differ from a native sequence by substitution, deletion or addition of five amino acids or fewer. Variants may also be modified by deletion or addition of amino acids, which have minimal influence on the immunogenicity, secondary structure and hydropathic nature of the polypeptide.

A variant may also mean a protein that is substantially identical to a reference protein.

p. “Vector”

“Vector” as used herein may mean a nucleic acid molecule capable of transporting another nucleic acid to which it has been linked, such as a plasmid. The vector may be capable of extra-chromosomal replication, such as an episome. The vector may be capable of directing expression of the nucleic acid to which it is operatively linked, such as an expression vector.

### 2. GLYCEROL UTILIZATION CELL

Provided herein is a microbial cell suitable for the bioconversion of glycerol. The cell may be a glycerol-utilizing cell. A glycerol utilization cell may take glycerol in and convert it to a target compound. The cell may be recombinantly produced. The cell may be a transformed cell comprising a glycerol-related nucleic acid sequence. The cell may be derived from any microbial cell including E. coli, Shigella dysenteriae, Shigella Flexneri, Shigella boydii, Salmonella enterica, Salmonella typhimurium, Enterobacter sp., Yersinia mollaretii, Yersinia bercovieri, Yersinisia pestis, Yersinia intermedia, Yersinia frederiksenii, Serratia proteamaculans, Erwinia carotovora, Pseudomonas fluorescens, Pseudomonas tolaasii, Salmonella enterica, Haemophilus influenzae, Pseudomonas putida, Pseudomonas syringae, Yersinia enterolitica, Photorhabdus luminesens, Axotobacter vinelandii, Pasteurella multocida, Shigella sonnei, Yersinia intermedia, Haemophilus ducreyi, Actinobacillus pleuropneumoniae, Aeromonas hydrophila, Photobacterium profundum, Aeromonas salmonicida, Vibrio angustum, Vibrio cholerae, Vibrio vulnificus, Vibrio fischeri, Vibrionales bacterium, Vibrio splendidus, Vibrio sp. Ex25, Vibrio harveyi, Vibrio alginolyticus, Vibrio parahaemolyticus, Shewanella sp. W3-18-1, Alteromonas macleodii, Sodalis glossinidius, Mycoplasma mycoides, Mycoplasma sp. ‘bovine group 7’, Mycoplasma capricolum, Mycoplasma agalactiae, Kluyveromyces lactis cell, Ashbya gossypii, Lodderomyces elongisporus, Debaryomyces hansenii, Candida albicans, Pichia guilliermondii, and Pichia stipitis.

The microbial cell may be

### 3. GLYCEROL RESISTANCE

The microbial cell may be resistant to toxicity associated with increased concentration of glycerol and/or increased glycerol metabolism. The microbial cell may have constitutive, unregulated expression of a glycerol regulon.

The glycerol resistant cell may be any species of bacterial cell capable of growing on compositions comprising glycerol. The cell may be a recombinant cell or a mutant cell selected for desirable growth characteristics in or on compositions comprising glycerol. The selection of an appropriate host is within the abilities of those skilled in the art. Examples of a glycerol resistant cell may include the E. coli strain deposited in the American Type Culture Collection as: (IV638653-39710; PTA-8496).

### 4. GLYCEROL METABOLIZING SYSTEM

The glycerol utilizing cell may be used in the production of end-product derivatives of glycerol. This cell may employ a glycerol metabolic system. The glycerol metabolic system may comprise a protein. The glycerol metabolic system may comprise a plurality of proteins. The glycerol metabolic system may comprise a glycerol kinase, glycerol dehydrogenase, glycerol dehydratase, 1,3-propanediol oxidoreductase, dihydroxyacetone (glycerone) kinase, alcohol dehydrogenase, alcohol dehydrogenase (NADP), D-glyceraldehyde dehydrogenase, glycerol-3-phosphate dehydrogenase (NAD(P)), 3-Phospho-D-glycerate dehydrogenase, glycerol-3-phosphate oxidase, glycerol oxidase, glycerol-1-phosphatase, propanediol-phosphate dehydrogenase, aldehyde reductase (dehydrogenase), aldehyde reductase (dehydrogenase) (NAD), glycerol dehydrogenase, 1,3-propanediol dehydrogenase, propanediol dehydratase, 1,3-propanediol dehydratase, and/or glycerate kinase.

Proteins of the glycerol metabolic system may be expressed in vitro or in vivo from a nucleic acid. The glycerol metabolic system may comprise a polypeptide sequence or a variant thereof or fragment thereof.

Under aerobic conditions the cell may convert intracellular glycerol into glycerol-3-phosphate via the enzyme, glycerol kinase. The glycerol-3-phosphate remains inside the cell, where it can be further metabolized. The glycerol kinase may have a propensity to associate with the cytoplasmic membrane. The glycerol kinase activity may be increased in vivo by the presence of a glycerol facilitator. Effective glycerol phosphorylation may rely on the interaction between the facilitator and the kinase.

Under anaerobic conditions, the cell may dissimilate free non-phosphorylated glycerol through coupled oxidative and reductive pathways. The oxidation of glycerol may be catalyzed by glycerol dehydrogenase, glycerol dehydratase, and/or 1,3-propanediol oxidoreductase. Numerous organisms possess genes encoding either a glycerol dehydratase and/or a 1,3-propanediol dehydratase that are expressed under anaerobic growth in glycerol. Dihydroxyacetone (glycerone) formed by glycerol dehydrogenase may be further metabolized to produce various compounds. These compounds may be 3-hydroxypropionaldehyde or 1,3-propanediol. Product formation may depend primarily on the availability of glycerol as a source of carbon and energy. Some specific enzymes are described more fully below.

a. Glycerol Kinase

The glycerol metabolic system may employ the use of a glycerol kinase. A glycerol kinase may be a polypeptide responsible for an enzyme activity that catalyzes the conversion of glycerol and ATP to glycerol-3-phosphate and ADP. The high-energy phosphate donor ATP may be replaced by physiological substitutes (e.g., phosphoenolpyruvate). Glycerol kinase is encoded, for example, by GUT1 (GenBank U11583×19) and glpK (GenBank L19201) (see WO 9928480, herein incorporated by reference).

b. Glycerol Dehydrogenase

The glycerol metabolic system may employ the use of a glycerol dehydrogenase. A glycerol dehydrogenase may be a polypeptide responsible for an enzyme activity that catalyzes the conversion of glycerol to dihydroxyacetone (E.C. 1.1.1.6) or glycerol to glyceraldehyde (E.C. 1.1.1.72). A polypeptide responsible for an enzyme activity that catalyzes the conversion of glycerol to dihydroxyacetone is also referred to as a “dihydroxyacetone reductase”. Glycerol dehydrogenase may be dependent upon NADH (E.C. 1.1.1.6), NADPH (E.C. 1.1.1.72), or other cofactors (e.g., E.C. 1.1.99.22). A NADH-dependent glycerol dehydrogenase is encoded, for example, by gldA (GenBank U00006) (see WO 9928480, herein incorporated by reference).

c. Glycerol Dehydratase

The glycerol metabolic system may employ the use of a dehydratase enzyme or a dehydratase. A dehydratase enzyme or a dehydratase may be responsible for any enzyme activity that catalyzes the conversion of a glycerol molecule to the product 3-hydroxypropionaldehyde. The dehydratase enzymes may include a glycerol dehydratase (E.C. 4.2.1.30) and a diol dehydratase (E.C. 4.2.1.28) and may have preferred substrates of glycerol and 1,2-propanediol, respectively. Genes for dehydratase enzymes have been identified in Klebsiella pneumoniae, Citrobacter freundii, Clostridium pasteurianum, Salmonella typhimurium, and Klebsiella oxytoca. In each case, the dehydratase is composed of three subunits: the large or “a” subunit, the medium or “β” subunit, and the small or “γ” subunit. The genes are described in, for example, Daniel et al. (FEMS Microbiol. Rev. 22, 553 (1999)) and Toraya and Mori (J. Biol. Chem. 274, 3372 (1999)).

d. 1,3-propanediol Oxidoreductase

The glycerol metabolic system may employ the use of a 1,3-propanediol oxidoreductase or a 1,3-propanediol dehydrogenase or “DhaT.” Each of 1,3-propanediol oxidoreductase or a 1,3-propanediol dehydrogenase or “DhaT” may be a polypeptide responsible for an enzyme activity that is capable of catalyzing the interconversion of 3-HPA and 1,3-propanediol. The gene(s) encoding such activity may be found to be physically or transcriptionally linked to a dehydratase enzyme in its natural (i.e., wild type) setting; for example, the gene may be found within a dha regulon as is the case with dhaT from Klebsiella pneumonia. Genes encoding a 1,3-propanediol oxidoreductase include dhaT from Klebsiella pneumoniae, Citrobacter freundii, and Clostridium pasteurianum. Each of these genes may encode a polypeptide belonging to the family of type III alcohol dehydrogenases.

e. Glycerol-3-phosphate Dehydrogenase

The glycerol metabolic system may employ the use of a glycerol-3-phosphate dehydrogenase or “G3PDH.” G3PDH may be a polypeptide having an enzyme activity that catalyzes the conversion of dihydroxyacetone phosphate (DHAP) to glycerol-3-phosphate (G3P). In vivo G3PDH may be NADH; NADPH; or FAD-dependent. When specifically referring to a cofactor specific glycerol-3-phosphate dehydrogenase, the terms “NADH-dependent glycerol-3-phosphate dehydrogenase”, “NADPH-dependent glycerol-3-phosphate dehydrogenase” and “FAD-dependent glycerol-3-phosphate dehydrogenase” may be used. As it is generally the case that NADH-dependent and NADPH-dependent glycerol-3-phosphate dehydrogenases may be able to use NADH and NADPH interchangeably (for example by the gene encoded by gpsA). The NADH-dependent enzyme (EC 1.1.1.8) may be encoded by several genes including GPD1 (GenBank Z74071×2), or GPD2 (GenBank Z35169×1), or GPD3 (GenBank G984182), or DAR1 (GenBank Z74071×2). The NADPH-dependent enzyme (EC 1.1.1.94) is encoded by gpsA (GenBank U321643, (cds 197911-196892) G466746 and L45246). The FAD-dependent enzyme (EC 1.1.99.5) may be encoded by GUT2 (GenBank Z47047×23), or glpD (GenBank G147838), or glpABC (GenBank M20938) (see WO 9928480 and references therein, which are herein incorporated by reference).

### 5. GLYCEROL UPTAKE PROTEINS

The glycerol utilizing cell may comprise glycerol uptake proteins. The cell may comprise nucleic acid encoding glycerol uptake proteins. Specific glycerol uptake proteins may be identified as ideal for the herein described method based upon analyses utilizing the ERGO bioinformatics database. The ERGO bioinformatics database may be used in in silico analyses of glycerol uptake nucleic acids and glycerol metabolism nucleic acids. In silico analyses may determine potential sites of bottlenecks in glycerol metabolism that may result from increased glycerol uptake from cloning glycerol uptake nucleic acids into a host cell.

The glycerol uptake protein may be a glycerol facilitator protein, a glycerol-specific ATP-dependent transporter protein, and/or a proton/glycerol symporter protein. The glycerol facilitator protein, or variant thereof, may be one selected from Table 1. The protein may be derived from E. coli, Shigella dysenteriae, Shigella Flexneri, Shigella boydii, Salmonella enterica, Salmonella typhimurium, Enterobacter sp., Yersinia mollaretii, Yersinia bercovieri, Yersinisia pestis, Yersinia intermedia, Yersinia frederiksenii, Serratia proteamaculans, Erwinia carotovora, Pseudomonas fluorescens, Pseudomonas tolaasii, Salmonella enterica, Haemophilus influenzae, Pseudomonas putida, Pseudomonas syringae, Yersinia enterolitica, Photorhabdus luminesens, Azotobacter vinelandii, Pasteurella multocida, Shigella sonnei, Yersinia intermedia, Haemophilus ducreyi, Actinobacillus pleuropneumoniae, Aeromonas hydrophila, Photobacterium profundum, Aeromonas salmonicida, Vibrio angustum, Vibrio cholerae, Vibrio vulnificus, Vibrio fischeri, Vibrionales bacterium, Vibrio splendidus, Vibrio sp. Ex25, Vibrio harveyi, Vibrio alginolyticus, Vibrio parahaemolyticus, Shewanella sp. W3-18-1, Alteromonas macleodii, or Sodalis glossinidius. The glycerol-specific ATP-dependent transporter protein, or variant thereof, may be one selected from Table 2. The protein may be derived from Mycoplasma mycoides, Mycoplasma sp. ‘bovine group 7’, Mycoplasma capricolum, or Mycoplasma agalactiae. The proton/glycerol symporter protein, or variant thereof, may be one selected from Table 3. The protein may be derived from Saccharomyces cerevisiae, Kluyveromyces lactis, Ashbya gossypii, Lodderomyces elongisporus, Debaryomyces hansenii, Candida albicans, Pichia guilliermondii, or Pichia stipitis.

### 6. GLYCEROL FACILITATOR PROTEIN

Glycerol uptake proteins may include glycerol facilitator proteins. Glycerol facilitator proteins may catalyze the facilitated diffusion of glycerol via an energy-independent process, whereby glycerol is transported into a bacterial cell expressing one or more of these proteins. A glycerol facilitator may be the E. coli protein GlpF (GenBank accession: NP_{—}418362.1).

Glycerol uptake proteins may also employ a variant of glycerol facilitator protein. The variant may have an amino acid sequence that is derived from the amino acid sequence of the precursor glycerol facilitator. The precursor glycerol facilitators include naturally-occurring glycerol facilitators and recombinant glycerol facilitators. The amino acid sequence of the glycerol facilitator variant may be derived from the precursor glycerol facilitator amino acid sequence by the substitution, deletion or insertion of one or more amino acids of the precursor amino acid sequence. Such modification may be of the precursor nucleic acid sequence which encodes the amino acid sequence of the precursor glycerol facilitator rather than manipulation of the precursor glycerol facilitator enzyme per se.

Glycerol uptake proteins may employ a glycerol facilitator protein that is substantially identical to the E. coli protein GlpF (GenBank accession: NP_{—}418362.1). A glycerol facilitator protein that is substantially identical to the E. coli protein GlpF may be one selected from TABLE 1.

a. Glycerol-Specific ATP-Dependent Transporter Protein

Glycerol uptake proteins may include a glycerol-specific ATP-dependent transporter protein. A glycerol-specific ATP-dependent transporter protein may be capable of active, ATP-dependent catalysis of glycerol transport into bacterial cells. A glycerol-specific ATP-dependent transporter may be Mycoplasma mycoides GtsA, GtsB and GtsC (NCBI CoreNucleotide accession AF251037).

Glycerol uptake proteins may also include a variant of a glycerol-specific energy-dependent transporter protein. The variant may have an amino acid sequence that is derived from the amino acid sequence of a precursor glycerol-specific ATP-dependent transporter. The precursor glycerol-specific ATP-dependent transporters may be naturally-occurring glycerol-specific ATP-dependent transporters or recombinant glycerol-specific ATP-dependent transporters. The amino acid sequence of the glycerol-specific ATP-dependent transporter variant may be derived from the precursor glycerol-specific ATP-dependent transporter amino acid sequence by the substitution, deletion or insertion of one or more amino acids of the precursor amino acid sequence. Such modification may be of the precursor nucleic acid sequence that encodes the amino acid sequence of the precursor glycerol-specific ATP-dependent transporter rather than manipulation of the precursor glycerol-specific ATP-dependent transporter enzyme per se.

Glycerol uptake proteins may include a glycerol-specific energy-dependent transporter protein that is substantially identical to the Mycoplasma mycoides GtsA, GtsB and GtsC (NCBI CoreNucleotide accession AF251037). A glycerol-specific ATP-dependent transporter protein that is substantially identical to the Mycoplasma mycoides GtsA, GtsB and GtsC may be one selected from TABLE 2.

b. Proton/Glycerol Symporter Protein

Glycerol uptake proteins may include a proton/glycerol symporter protein. encompasses enzymes capable of conferring onto a cell the ability to take up glycerol against a concentration gradient in a proton motive force-dependent manner. An example of such a proton/glycerol symporter is the protein encoded by the S. cerevisiae gene STL1 (GenBank accession: NP_{—}010825.1).

Glycerol uptake proteins may include a proton/glycerol symporter variant protein. The variant may have an amino acid sequence that is derived from the amino acid sequence of a precursor proton/glycerol symporter. The precursor proton/glycerol symporters may include naturally-occurring proton/glycerol symporters and recombinant proton/glycerol symporters. The amino acid sequence of the proton/glycerol symporter variant may be derived from the precursor proton/glycerol symporter amino acid sequence by the substitution, deletion or insertion of one or more amino acids of the precursor amino acid sequence. Such modification may be of the precursor nucleic acid sequence which encodes the amino acid sequence of the precursor proton/glycerol symporter rather than manipulation of the precursor proton/glycerol symporter enzyme per se.

Glycerol uptake proteins may include a proton/glycerol symporter protein that is substantially identical to the S. cerevisiae protein STL1 (GenBank accession: NP_{—}010825.1). A proton/glycerol symporter protein that is substantially identical to the S. cerevisiae protein STL1 may be one selected from TABLE 3.

TABLE 1
Facilitator Protein

| | | % identity to E. coli |
|---|---|---|
| | | glpF protein (281 |
| Accession Number | Organism/Protein Name | amino acids) |
| NP_290556.1 | E. coli o157: H7/glycerol facilitator protein | 100 |
| ZP_00921256.1 | Shigella dysenteriae 1012/glycerol uptake | 99 |
| | facilitator | |
| ref\|YP_405253.1 | Shigella dysenteriae Sd197/facilitated diffusion | 99 |
| | of glycerol | |
| sp\|P31140 | Shigella flexneri/glycerol uptake facilitator | 99 |
| | protein | |
| ref\|ZP_00707869.1 | E. coli HS/glycerol uptake facilitator | 99 |
| ref\|YP_410223.1 | Shigella boydii Sb227/facilitated diffusion of | 99 |
| | glycerol | |
| gb\|AAA21363.1 | E. coli HB101/glycerol diffusion facilitator | 98 |
| | protein | |
| ref\|ZP_00696814.1 | Shigella boydii BS512/glycerol uptake | 99 |
| | facilitator | |
| emb\|CAA33153.1 | E. coli K12/unnamed | 99 |
| emb\|CAA77815.2 | Shigella flexneri/glpF | 99 |
| pdb\|1LDF\|A | E. coli/glycerol facilitator(W48F, F200T) | 99 |
| ref\|NP_457965.1 | Salmonella enterica/glycerol uptake facilitator | 93 |
| | protein | |
| ref\|NP_462968.1 | Salmonella typhimurium LT2/glycerol | 92 |
| | diffusion | |
| gb\|AAO59936.1 | Uncultured bacterium/GlpF | 90 |
| ref\|YP_001178752.1 | Enterobacter sp. 638/MIP family channel | 88 |
| | protein | |
| ref\|ZP_00824891.1 | Yersinia mollaretii ATCC 43969/glycerol | 84 |
| | uptake facilitator | |
| ref\|NP_403753.1 | glycerol uptake facilitator protein [Yersinia | 82 |
| | pestis CO92] | |
| ref\|ZP_00821450.1 | Glycerol uptake facilitator and related | 85 |
| | permeases (Major | |
| | Intrinsic Protein Family) [Yersinia bercovieri | |
| | ATCC 43970] | |
| ref\|YP_001004494.1 | glycerol uptake facilitator protein [Yersinia | 85 |
| | enterocolitica | |
| | subsp. enterocolitica 8081] | |
| ref\|ZP_00832843.1 | Glycerol uptake facilitator and related | 86 |
| | permeases (Major | |
| | Intrinsic Protein Family) [Yersinia intermedia | |
| | ATCC 29909] | |
| ref\|ZP_00828528.1 | Glycerol uptake facilitator and related | 86 |
| | permeases (Major | |
| | Intrinsic Protein Family) [Yersinia frederiksenii | |
| | ATCC | |
| | 33641] | |
| ref\|ZP_01537482.1 | MIP family channel proteins [Serratia | 81 |
| | proteamaculans 568] | |
| ref\|YP_052353.1 | glycerol uptake facilitator protein [Erwinia | 81 |
| | carotovora subsp. | |
| | atroseptica SCRI1043] | |
| ref\|NP_709731.1 | glycerol diffusion facilitator protein [Shigella | 100 |
| | flexneri 2a | |
| | str. 301] | |
| ref\|YP_261948.1 | glycerol uptake facilitator protein | 71 |
| | [Pseudomonas fluorescens | |
| | Pf-5] | |
| dbj\|BAA31549.1 | glpF [Pseudomonas tolaasii] | 71 |
| ref\|YP_217032.1 | Propanediol utilization: propanediol diffusion | 69 |
| | facilitator [Salmonella | |
| | enterica subsp. enterica serovar Choleraesuis | |
| | str. | |
| | SC-B67] | |
| ref\|ZP_00154555.2 | Glycerol uptake facilitator and related | 73 |
| | permeases (Major | |
| | Intrinsic Protein Family) [Haemophilus | |
| | influenzae R2846] | |
| ref\|ZP_01797866.1 | glycerophosphodiester phosphodiesterase | 73 |
| | [Haemophilus influenzae | |
| | R3021] | |
| ref\|YP_001172131.1 | glycerol uptake facilitator protein | 71 |
| | [Pseudomonas stutzeri A1501] | |
| ref\|NP_438850.1 | glycerol uptake facilitator protein | 73 |
| | [Haemophilus influenzae Rd | |
| | KW20] | |
| ref\|ZP_00156488.1 | Glycerol uptake facilitator and related | 73 |
| | permeases (Major | |
| | Intrinsic Protein Family) [Haemophilus | |
| | influenzae R2866] | |
| ref\|ZP_01785415.1 | glycerol kinase [Haemophilus influenzae 22.1- | 73 |
| | 21] | |
| ref\|YP_248379.1 | glycerol uptake facilitator protein | 73 |
| | [Haemophilus influenzae 86-028NP] | |
| ref\|ZP_01639665.1 | MIP family channel proteins [Pseudomonas | 74 |
| | putida W619] | |
| ref\|YP_276035.1 | glycerol uptake facilitator protein | 70 |
| | [Pseudomonas syringae pv. | |
| | phaseolicola 1448A] | |
| ref\|YP_350259.1 | Aquaporin [Pseudomonas fluorescens PfO-1] | 72 |
| ref\|YP_236972.1 | Major intrinsic protein [Pseudomonas syringae | 71 |
| | pv. syringae B728a] | |
| ref\|YP_001006918.1 | propanediol diffusion facilitator [Yersinia | 66 |
| | enterocolitica subsp. | |
| | enterocolitica 8081] | |
| ref\|ZP_00831359.1 | Glycerol uptake facilitator and related | 66 |
| | permeases (Major | |
| | Intrinsic Protein Family) [Yersinia frederiksenii | |
| | ATCC | |
| | 33641] | |
| ref\|NP_793928.1 | glycerol uptake facilitator protein | 70 |
| | [Pseudomonas syringae pv. | |
| | tomato str. DC3000] | |
| ref\|YP_606904.1 | glycerol uptake facilitator protein | 74 |
| | [Pseudomonas entomophila | |
| | L48] | |
| ref\|NP_743237.1 | glycerol uptake facilitator protein | 73 |
| | [Pseudomonas putida KT2440] | |
| ref\|ZP_00826241.1 | Glycerol uptake facilitator and related | 68 |
| | permeases (Major | |
| | Intrinsic Protein Family) [Yersinia mollaretii | |
| | ATCC 43969] | |
| ref\|ZP_01716496.1 | MIP family channel proteins [Pseudomonas | 73 |
| | putida GB-1] | |
| ref\|ZP_00823346.1 | Glycerol uptake facilitator and related | 67 |
| | permeases (Major | |
| | Intrinsic Protein Family) [Yersinia bercovieri | |
| | ATCC 43970] | |
| ref\|NP_931927.1 | glycerol uptake facilitator protein | 67 |
| | [Photorhabdus luminescens | |
| | subsp. laumondii TTO1] | |
| ref\|ZP_00419186.1 | Major intrinsic protein [Azotobacter vinelandii | 72 |
| | AvOP] | |
| ref\|NP_246384.1 | GlpF [Pasteurella multocida subsp. multocida | 70 |
| | str. Pm70] | |
| ref\|ZP_01293105.1 | hypothetical protein PaerP_01005015 | 74 |
| | [Pseudomonas aeruginosa PA7] | |
| ref\|NP_460982.1 | propanediol diffusion facilitator [Salmonella | 69 |
| | typhimurium LT2] | |
| ref\|NP_456587.1 | propanediol diffusion facilitator [Salmonella | 68 |
| | enterica subsp. | |
| | enterica serovar Typhi str. CT18] | |
| ref\|NP_252271.1 | glycerol uptake facilitator protein | 74 |
| | [Pseudomonas aeruginosa PAO1] | |
| emb\|CAJ87611.1 | putative propanediol diffusion facilitator | 69 |
| | [Escherichia coli] | |
| gb\|AAB57803.1 | glycerol diffusion facilitator [Pseudomonas | 74 |
| | aeruginosa] | |
| ref\|ZP_00700062.1 | Glycerol uptake facilitator and related | 67 |
| | permeases (Major | |
| | Intrinsic Protein Family) [Escherichia coli | |
| | E24377A] | |
| ref\|YP_310948.1 | propanediol diffusion facilitator [Shigella | 70 |
| | sonnei Ss046] | |
| ref\|ZP_00832767.1 | Glycerol uptake facilitator and related | 68 |
| | permeases (Major | |
| | Intrinsic Protein Family) [Yersinia intermedia | |
| | ATCC 29909] | |
| ref\|NP_873618.1 | glycerol uptake facilitator protein | 70 |
| | [Haemophilus ducreyi 35000HP] | |
| gb\|AAB84108.1 | PduF [Salmonella enterica subsp. enterica | 67 |
| | serovar Typhimurium] | |
| ref\|YP_001053083.1 | glycerol uptake facilitator protein | 72 |
| | [Actinobacillus pleuropneumoniae | |
| | L20] | |
| ref\|YP_856185.1 | glycerol uptake facilitator protein [Aeromonas | 66 |
| | hydrophila subsp. | |
| | hydrophila ATCC 7966] | |
| ref\|YP_128490.1 | putative glycerol uptake facilitator protein | 65 |
| | [Photobacterium | |
| | profundum SS9] | |
| ref\|ZP_01221619.1 | putative glycerol uptake facilitator protein | 64 |
| | [Photobacterium | |
| | profundum 3TCK] | |
| ref\|YP_001142476.1 | glycerol uptake facilitator [Aeromonas | 68 |
| | salmonicida subsp. salmonicida | |
| | A449] | |
| ref\|ZP_01234850.1 | glycerol uptake facilitator protein GlpF [Vibrio | 66 |
| | angustum S14] | |
| ref\|ZP_01480174.1 | hypothetical protein VchoM_02000705 [Vibrio | 67 |
| | cholerae MO10] | |
| ref\|ZP_01161041.1 | glycerol uptake facilitator protein GlpF | 66 |
| | [Photobacterium sp. | |
| | SKA34] | |
| ref\|ZP_01679138.1 | glycerol uptake facilitator protein [Vibrio | 66 |
| | cholerae V52] | |
| ref\|ZP_01482916.1 | hypothetical protein VchoR_02001180 [Vibrio | 67 |
| | cholerae RC385] | |
| ref\|NP_760670.1 | Glycerol uptake facilitator [Vibrio vulnificus | 65 |
| | CMCP6] | |
| ref\|YP_206193.1 | glycerol uptake facilitator protein [Vibrio | 65 |
| | fischeri ES114] | |
| gb\|EDK31071.1 | glycerol uptake facilitator protein GlpF | 64 |
| | [Vibrionales bacterium | |
| | SWAT-3] | |
| ref\|ZP_00989512.1 | glycerol uptake facilitator protein GlpF [Vibrio | 65 |
| | splendidus 12B01] | |
| ref\|ZP_01475189.1 | hypothetical protein VEx2w_02002255 [Vibrio | 66 |
| | sp. Ex25] | |
| gb\|EDL68672.1 | glycerol uptake facilitator protein GlpF [Vibrio | 65 |
| | harveyi HY01] | |
| ref\|ZP_01258661.1 | glycerol uptake facilitator protein GlpF [Vibrio | 65 |
| | alginolyticus | |
| | 12G01] | |
| ref\|NP_798764.1 | glycerol uptake facilitator protein GlpF [Vibrio | 65 |
| | parahaemolyticus | |
| | RIMD 2210633] | |
| ref\|YP_961830.1 | MIP family channel protein [Shewanella sp. | 62 |
| | W3-18-1] | |
| ref\|ZP_01109118.1\| | glycerol uptake facilitator protein GlpF | 61 |
| | [Alteromonas macleodii | |
| | ‘Deep ecotype’] | |
| ref\|YP_455538.1 | propanediol diffusion facilitator [Sodalis | 61 |
| | glossinidius str. | |
| | ‘morsitans’] | |

TABLE 2
Glycerol-Specific ATP-Dependent Transporter Protein

| | | % identity to Mycoplasma |
|---|---|---|
| | | mycoides gtsABC multi- |
| | | protein (NCBI |
| | | CoreNucleotide Accession |
| Accession Number | Protein Name/Organism | No. AF251037) |
| gb\|AAG41804.1\|AF251037_1 | glycerol transporter subunit | 100 (subunit A) |
| | A [Mycoplasma mycoides | |
| | subsp. mycoides | |
| | SC] | |
| ref\|NP_975502.1\| | ABC transporter, ATP- | 99 (subunit A) |
| | binding component | |
| | [Mycoplasma mycoides | |
| | subsp. mycoides SC str. | |
| | PG1] | |
| emb\|CAD12044.1\| | glycerol transporter subunit | 96 (subunit A) |
| | A [Mycoplasma sp. ‘bovine | |
| | group | |
| | 7’] | |
| ref\|YP_424428.1\| | glycerol ABC transporter, | 81 (ATP-binding |
| | ATP-binding protein | component) |
| | [Mycoplasma capricolum | |
| | subsp. capricolum ATCC | |
| | 27343] | |
| emb\|CAD12045.1\| | glycerol transporter subunit | 96 (subunit B) |
| | B [Mycoplasma sp. ‘bovine | |
| | group | |
| | 7’] | |
| ref\|NP_975503.1\| | Glycerol ABC transporter | 100 (subunit B-permease) |
| | subunit B, permease | |
| | component [Mycoplasma | |
| | mycoides | |
| | subsp. mycoides SC str. | |
| | PG1] | |
| gb\|AAF24205.1\|AF165135_5 | hypothetical ABC | 100 (ABC transporter |
| | transporter protein | protein) |
| | [Mycoplasma mycoides | |
| | subsp. | |
| | mycoides SC] | |
| gb\|AAF24200.1\|AF165134_1 | hypothetical ABC | 100 (ATP-binding |
| | transporter ATP-binding | component) |
| | protein [Mycoplasma | |
| | mycoides subsp. mycoides | |
| | SC] | |
| ref\|YP_001256373.1\| | Glycerol ABC transporter, | 62 (ATP-binding |
| | ATP-bindingcomponent | component) |
| | [Mycoplasma agalactiae | |
| | PG2] | |
| ref\|YP_424427.1\| | glycerol ABC transporter, | 86 (permease) |
| | permease [Mycoplasma | |
| | capricolum subsp. | |
| | capricolum ATCC 27343] | |
| ref\|NP_975504.1\| | Glycerol ABC transporter, | 100 (subunit C) |
| | permease component | |
| | [Mycoplasma mycoides | |
| | subsp. mycoides SC str. | |
| | PG1] | |
| ref\|YP_424426.1\| | glycerol ABC transporter, | 86 (permease) |
| | permease [Mycoplasma | |
| | capricolum subsp. | |
| | capricolum ATCC 27343] | |
| emb\|CAD12046.1\| | glycerol transporter subunit | 88 (subunit C) |
| | C [Mycoplasma sp. ‘bovine | |
| | group | |
| | 7’] | |
| ref\|YP_001256374.1\| | Glycerol ABC transporter, | 64 (permease) |
| | permease component | |
| | [Mycoplasma agalactiae | |
| | PG2] | |
| ref\|YP_001256793.1\| | Glycerol transporter subunit | 65 (subunit B) |
| | B [Mycoplasma agalactiae | |
| | PG2] | |
| ref\|YP_001256792.1\| | Glycerol ABC transporter, | 71 (permease) |
| | permease component | |
| | [Mycoplasma agalactiae | |
| | PG2] | |

TABLE 3
Proton/Glycerol Symporter Protein.

| | | % identity to |
|---|---|---|
| | | S. cerevisiae |
| | | STL1 protein |
| Accession Number | Protein Name/Organism | (569 amino acids) |
| ref\|NP_010825.1 | Glycerol proton symporter | 100 |
| | of the plasma membrane, | |
| | subject to | |
| | glucose-induced | |
| | inactivation, strongly but | |
| | transiently induced | |
| | when cells are subjected to | |
| | osmotic shock | |
| | [Saccharomyces | |
| | cerevisiae] | |
| gb\|AAU09713.1 | YDR536W [Saccharomyces | 99 |
| | cerevisiae] | |
| gb\|AAA57229.1 | Sugar transport protein | 100 |
| | [Saccharomyces cerevisiae] | |
| ref\|XP_456249.1 | unnamed protein product | 72 |
| | [Kluyveromyces lactis] | |
| ref\|NP_984235.1 | ADR139Cp [Ashbya | 69 |
| | gossypii ATCC 10895] | |
| gb\|EDK46768.1 | sugar transporter STL1 | 63 |
| | [Lodderomyces | |
| | elongisporus NRRL YB- | |
| | 4239] | |
| ref\|XP_459387.1 | hypothetical protein | 61 |
| | DEHA0E01936g | |
| | [Debaryomyces hansenii | |
| | CBS767] | |
| ref\|XP_718089.1 | hypothetical protein | 60 |
| | CaO19_5753 [Candida | |
| | albicans SC5314] | |
| ref\|XP_001483277.1 | hypothetical protein | 60 |
| | PGUG_04006 [Pichia | |
| | guilliermondii ATCC 6260] | |
| ref\|XP_001383774.1 | sugar transporter [Pichia | 61 |
| | stipitis CBS 6054] | |
| ref\|XP_001484307.1 | hypothetical protein | 62 |
| | PGUG_03688 [Pichia | |
| | guilliermondii ATCC 6260] | |

### 7. NUCLEIC ACID

Also provided herein is a nucleic acid that encodes a glycerol uptake protein or a variant thereof. The nucleic acid may encode a glycerol facilitator protein from Table 1. The nucleic acid may encode a glycerol-specific energy-dependent transporter protein from Table 2. The nucleic acid may encode a proton/symporter protein from Table 3. A nucleic acid may also encode a protein component of the glycerol metabolic system or a variant thereof. The nucleic acid may also encode a glycerol resistance gene or a variant thereof. The nucleic acid encoding a glycerol resistance gene, or variant thereof, may be derived from the cell deposited in ATCC (ATCC as E. coli K12:MG1655-R3/1 as identifier IV638653-39710; ATCC No. PTA-8496)). The nucleic acid may comprise native sequences such as an endogenous sequence.

The nucleic acid may be combined with other nucleic acid sequences, such as promoters, polyadenylation signals, polyhistidine signals additional restriction enzyme sites, multiple cloning sites, other coding segments, and the like. Nucleic acids may also be capable of hybridizing under moderately stringent conditions and specifically binding to a nucleic of an agent.

a. Vector

Also provided herein is a vector that comprises the nucleic acid. The vector may be an expression vector. The vector may comprise a nucleic acid sequence or plurality thereof encoding the amino acid sequences. The vector may express the nucleic acid in a heterologous expression alone or in combination with a cell's expression of endogenous genes.

The expression vector may include one or more control sequences capable of effecting and/or enhancing the expression of the agent. Control sequences that are suitable for expression in prokaryotes, for example, include a promoter sequence, an operator sequence, and a ribosome binding site. Control sequences for expression in eukaryotic cells may include a promoter, an enhancer, and a transcription termination sequence (i.e. a polyadenylation signal).

The expression vector may also include other sequences, such as, for example, nucleic acid sequences encoding a signal sequence or an amplifiable gene. A signal sequence may direct the secretion of a polypeptide fused thereto from a cell expressing the protein. In the expression vector, nucleic acid encoding a signal sequence may be linked to a polypeptide coding sequence so as to preserve the reading frame of the polypeptide coding sequence.

### 8. GLYCEROL UTILIZATION METHODS

Provided herein is a method of utilizing glycerol as a substrate for the production of functional end-product derivatives. The method allows for the bioconversion of pure glycerol or glycerol in the presence of contaminating substances.

The method may comprise providing a glycerol-utilizing cell that comprises a glycerol metabolizing system and a glycerol uptake protein. The host cell may be a glycerol utilization strain. The host cell may be manipulated in order to inactivate competing pathways for carbon flow by deleting various genes. This may require the availability of either transposons to direct inactivation or chromosomal integration vectors. The host cell may be amenable to chemical mutagenesis.

Host cell culture conditions may allow transcription, translation, and protein transport between cellular compartments. Factors that affect these processes are well-known and include, for example, DNA/RNA copy number; factors that stabilize nucleic acid; nutrients, supplements, and transcriptional inducers or repressors present in the culture medium; temperature, pH and osmolarity of the culture; and cell density. The adjustment of these factors to promote expression in a particular vector-host cell system is within the level of skill in the art. Nucleic acid may be used to synthesize or express (in vitro or in vivo) a component of the glycerol metabolizing system and/or a glycerol uptake protein. Principles and practical techniques for maximizing the productivity of microbial fermentation cultures, for example, may be found in Methods in Biotechnology No. 18: Microbial Processes and Products (Barredo, J.-L. Ed., Humana Press (2005)).

Any of a number of well-known techniques for large- or small-scale production of proteins may be employed in expressing a nucleic acid and production of a target compound. These may include the use of a shaken flask, a fluidized bed bioreactor, a roller bottle culture system, and a stirred tank bioreactor system. The cell culture may be cultured in a batch, fed-batch, or continuous mode.

### 9. GLYCEROL COMPOSITIONS

The herein described composition and/or method may comprise contacting the glycerol-utilizing cell with a glycerol composition. The herein described composition may comprise a glycerol-utilizing cell and a glycerol composition. The glycerol composition may be any solution or substrate comprising glycerol. The glycerol solution or substrate may be crude glycerol. The crude glycerol may be a biodiesel by-product.

a. Crude Glycerol

The method may employ a crude glycerol composition. A crude glycerol composition may be a solution comprising glycerol, wherein the solution is not 100% pure glycerol. A crude glycerol composition may be a composition having a glycerol purity of less than 100% relative to a contaminating component. A glycerol composition may have one or more components that are not glycerol (i.e. a contaminating component). A crude glycerol composition having 98%-99% glycerol may be referred to as technical grade glycerol.

Crude glycerol may be used directly or indirectly in the method. For indirect use, and prior to employing the crude glycerol in the method, impurities may be removed by conventional separation techniques to provide a higher concentration of glycerol in the solution or in/on the substrate.

(1) Biodiesel By-Product

The crude glycerol composition may be a by-product from the manufacture of biodiesel. The manufacture of biodiesel, and a by-product, may result from transesterification reactions with triglycerides. The by-product may be a composition that comprises between 50% and 95% glycerol, between 55% and 90%, between 60% and 85%, between 65% and 85%, 70% and 80%, or 75% and 80% glycerol. The composition may further comprise an enal compound, a triglyceride, a monoglyceride, an animal fat, a vegetable oil, a base, an alkaline base catalyst, a methyl ester of a fatty acid, ash, a soap, and/or an alcohol. The enal product may be a dehydration product of a glycerol compound. The enal compound may be an acrolein, or 2-propenal, or a polymer thereof. The soap may be an alkaline salt of a fatty acid. The alkaline salt may be a sodium or potassium salt. The alkaline base catalyst may be a sodium methoxide, sodium hydroxide, and/or potassium hydroxide. The ash may contain calcium potassium, magnesium sodium, phosphorus, and/or sulfur. The composition may have a pH of greater than 8, 8.5, 9, 9.5, 10, 10.5, 10.5, 11, or 11.5. The base may be a potassium salt. The alcohol may be an aliphatic alcohol. The aliphatic alcohol may be a methanol and/or an ethanol.

Glycerol-containing by-product may be used directly or indirectly in the method. For indirect use, and prior to employing the biodiesel by-product in the method, impurities may be removed by conventional separation techniques to provide a higher concentration of glycerol in the by-product stream.

### 10. TARGET COMPOUNDS

The method may be used to produce a number of target compounds. A glycerol-derivable target compound may be produced. A target compound may be propionic acid, ethanol, 1,3-propanediol, 1,2-propanediol, 3-hydroxypropionic acid, poly (3-hydroxy-butyrate), poly (3-mercapto-propionate), hydrogen, succinate, dihydroxyacetone, butyric acid, acetic acid, polyglutamic acid, cinnamic acid, rhamnolipids, 3-hydroxacetone, omega-3 polyunsaturated fatty acids, malate, oxaloacetate, fumarate, aconitate, citrate, isocitrate, 2-ketoglutarate, glycerol-3-phosphate, pyruvate, L-lactate, D-lactate, formate. In addition, amino acids, nucleobases, vitamins, antibiotics, and/or propylene glycol may be produced.

The biological production of 1,3-propanediol may require a glycerol composition as a substrate for a two-step sequential reaction in which a dehydratase enzyme (typically a coenzyme B₁₂-dependent dehydratase) converts glycerol to an intermediate, 3-hydroxypropionaldehyde, which is then reduced to 1,3-propanediol by a NADH- (or NADPH) dependent oxidoreductase. The reactions may require a cofactor and/or a whole cell catalyst for an industrial process that utilizes this reaction sequence for the production of 1,3-propanediol.

### 11. ISOLATING TARGET COMPOUNDS

Methods for recovery of the target compound(s) are well-known and vary depending on the cell culture system employed. The target compound may be produced intracellularly and recovered from cell lysates.

The target compound may be purified from culture medium or a cell lysate by any method capable of separating the compound from one or more components of the host cell or culture medium. The compound may be separated from host cell and/or culture medium components that would interfere with the intended use of the compound. As a first step, the culture medium or cell lysate may be centrifuged or filtered to remove cellular debris. The supernatant may then typically concentrated or diluted to a desired volume or diafiltered into a suitable buffer to condition the preparation for further purification.

The compound may then be further purified using well-known techniques. The technique chosen will vary depending on the properties of the compound.

### 12. METHODS FOR PURIFYING TARGET COMPOUND 1,3-propanediol

Methods for purifying 1.3 propanediol from fermentation media are known in the art. Propanediols may be obtained from cell media by subjecting the reaction mixture to extraction with an organic solvent, distillation and column chromatography (U.S. Pat. No. 5,356,812). A particularly good organic solvent for this process may be cyclohexane (U.S. Pat. No. 5,008,473).

1,3-Propanediol may be identified directly by submitting the media to high pressure liquid chromatography (HPLC) analysis. Fermentation media may be analyzed on an analytical ion exchange column using a mobile phase of 0.01N sulfuric acid in an isocratic fashion.

For industrial applications, purification of 1,3-propanediol from large volumes of fermentor broth may require non-laboratory scale methods. Difficulties to be overcome include removal of cell matter form the broth (clarification), concentration of 1,3-propanediol either by extraction or water removal and separation of residual impurities from the partially purified monomer.

Broth clarification may proceed either by filtration, centrifugation or crossflow microfiltration. Suitable filters are manufactured for example by Millipore (Millipore Corporation, 80 Ashby Road, Bedford, Mass.) or Filmtec (Dow Chemical Co.). Centrifugation effectively removes the bulk of the cells, but, depending upon the nature of the broth, does not always achieve complete cell removal. Crossflow microfiltration yields extremely clear filtrate. The concentrate is a slurry rather than a high-solids cake. The skilled person will be able to adapt the clarification method most appropriate for the fermentation apparatus and conditions being employed.

Water reduction of the clarified broth may be complicated by the high solubility of 1,3-propanediol in water. Extraction of 1,3-propanediol from the clarified broth may be accomplished by a variety of methods, including evaporation/distillation, membrane technology, extraction by organic solvent and adsorption.

Rotary evaporators may be used to initially reduce water volume in the clarified broth. This method has enjoyed good success in Applicants' hands. Precipitation of extraneous proteins and salts do not appear to affect 1,3-propanediol recovery.

Membrane technology may be used either separately or in conjunction with evaporation. Suitable membranes will either (i) allow passage of 1,3-propanediol, retaining water and other feed molecules (ii) allow passage of water and other molecules, retaining 1,3-propaned iol or (iii) allow passage of water and 1,3-propanediol while retaining other molecules. Particularly useful, are reverse osmosis membranes such as SW-30 2540 (Filmtec, Dow Chemical Co.) and the DL and SH series of reverse osmosis membranes made by Millipore (Millipore Corporation, Bedford, Mass.).

Following evaporation and membrane concentration, partially purified 1,3-propanediol may be extracted into organic solvents. Suitable solvent will include alcohols such as tert-amyl alcohol, cyclopentanol, octanol, propanol, methanol, and ethanol. Non alcohols may also be used such as octanone, cyclohexane and valeraldehyde.

1,3-propanediol may be further concentrated by adsorption to various industrial adsorbents. Activated carbon and polycyclodextrin such as those produced by the American Maize Products Company may be suitable.

Following either extraction or adsorption, partially purified 1,3-propanediol must be refined. Refining may be accomplished by electrodialysis (particularly useful for desalting) which utilizes a combination of anion and cation exchange membranes or biopolar (anion and cation) membranes (see for example, Grandison, Alistair S., Sep. Processes Food Biotechnol. Ind. (1996), 155 177.)

Refining may be accomplished via distillation. Distillation may be done in batch where the operating pressure is ambient or below, e.g. about 25 in. Hg of vacuum. Monitoring of distillation indicated that materials evaporated in the order of first to last beginning with light organics.

### EXAMPLES

### Example 1

### Selection for Glycerol Resistant E. coli

To select for Gly^{R} mutants, the Escherichia coli MG1655 cell sample was inoculated (10⁴ to 10⁵ cells) into M9 salt medium (Maniatis et al, 1989) containing 0.5% glycerol and 0.1 μg/ml of thiamine. 2.0% Bacto-Agar was added. A sample of E. coli MG1655 was mutagenized with nitrosoguanidine, using previously described protocols (Miller, 1972). The E. coli cultures to be mutagenized, were administered at a dose of N-methyl-N′-nitro-N-nitrosoguanidine sufficient to kill 50 to 90% of the cells. Log-phase cultures were washed twice in sodium citrate buffer, pH 5.5, incubated with 150 μg/ml of the mutagen for 15 to 30 min, washed twice in M9 minimal salts, and titered for cell survival. Mutagenized cultures were outgrown 1:20 into LB overnight cultures and processed for Gly^{R} mutants the following day. These mutagenized cells were then grown in a minimal M9 medium, which contained 12% glycerol until turbidity (OD₆₀₀ₙₘ) developed.

Serial dilutions of the cells were then plated onto solid M9 agar containing 12% glycerol and incubated at 37° C. to produce colonies. Wild type cells did not produce colonies on the agar plates with 12% of glycerol even after prolonged incubation. Candidate Gly^{R} cells were then streaked out on fresh M9 plates with 12% glycerol. Single colonies of glycerol resistant mutants were purified twice on the same agar. After analyzing a few dozen of chemically-induced mutants, strains were isolated with the ability to grow under aerobic growth conditions in the presence of 12% glycerol. Several mutants were obtained and characterized further.

### Example 2

### ATCC Deposited Strain that is Capable of Growing in High Levels of Glycerol

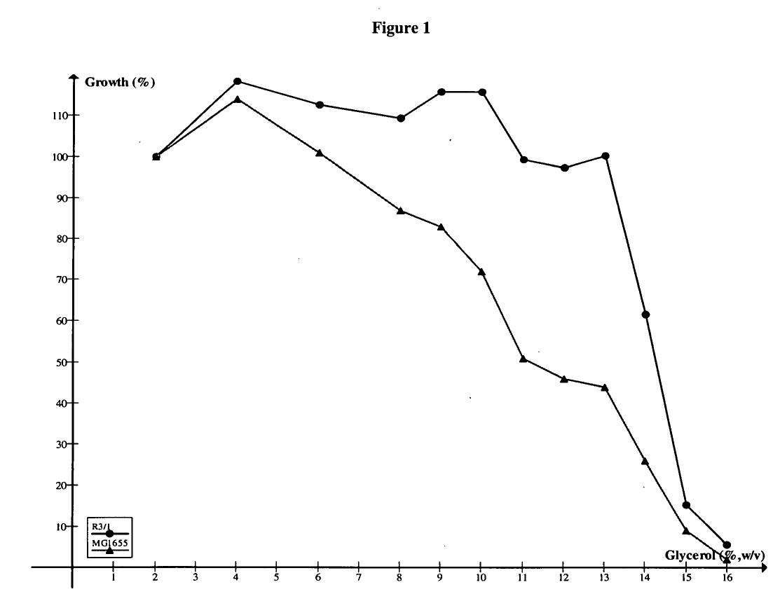

A mutant strain designated as MG1655-R3/1 (deposited in ATCC as E. coli K12 MG1655-R3/1 under identifier: IV638653-39710; ATCC No. PTA-8496)), acquired resistance to high concentrations of glycerol (indicated in FIG. 1 by the shoulder in the R3/1 mutant curve, red circles, compared to wild-type). Moreover, the mutant produces more biomass then parent organism even at relatively low concentrations of glycerol.

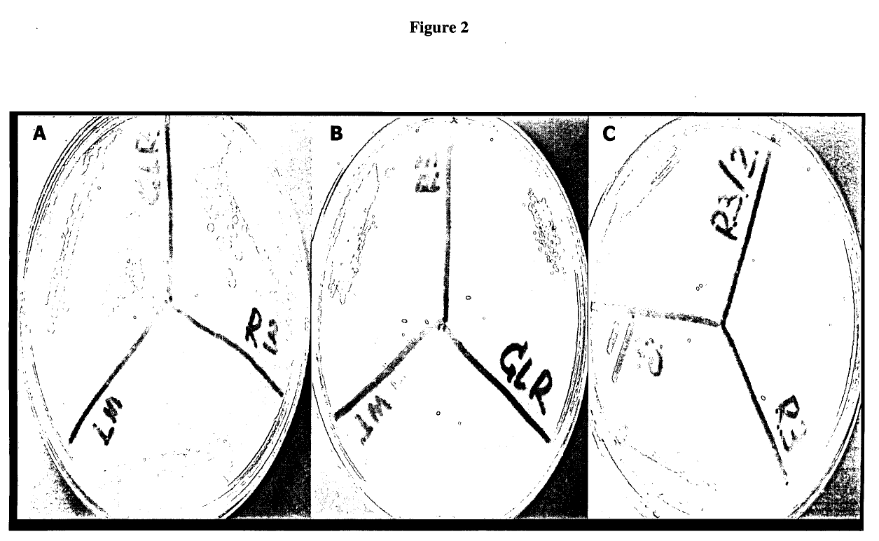

The physical growth characteristics of the mutant and wild-type strains were also compared on solid M9 minimal medium plates containing various concentrations of pure glycerol (see FIG. 2). Some of the mutants (e.g. designated R3 and GLR) grow better than wild-type (WT) cells (based on relative colony size) at 4.5% (FIG. 2A) and 9% glycerol (FIG. 2B). In the latter case i.e. 9% glycerol (FIG. 2B), wild-type cells are not growing compared to the mutant strains. Furthermore, additional mutants were isolated from the R3 mutant strain that demonstrated better growth properties on glycerol than the initial R3 mutant strain (see FIG. 2B; mutants R3/1 and R3/2). Thus, the isolation of the mutants described above has been performed and these strains may show significantly enhanced growth properties on glycerol compared to wild-type E. coli.

### Example 3

### Glycerol Uptake by ATCC Deposited Strain

Bacteria were grown in M9 medium containing glycerol (1%) and casamino acids (0.01%) until microbial turbidity reached OD₆₀₀ 0.8. Cells were harvested by centrifugation, washed out by M9 salt and resuspended in M9 salt to obtain optical density of cells around 6. Glycerol was added to a cell suspension to a final concentration of 1%. These samples were placed in an incubator (37° C.) with shaking (250 rpm). Two parallel samples were taken to measure optical density and glycerol concentration in the supernatant after cells were removed by centrifugation. The glycerol concentration was determined using a free glycerol reagent kit (Sigma) as directed by the manufacturer's instructions. The results are presented as micrograms (μg) of glycerol per ml of incubation medium divided by OD600 of the cell suspension.

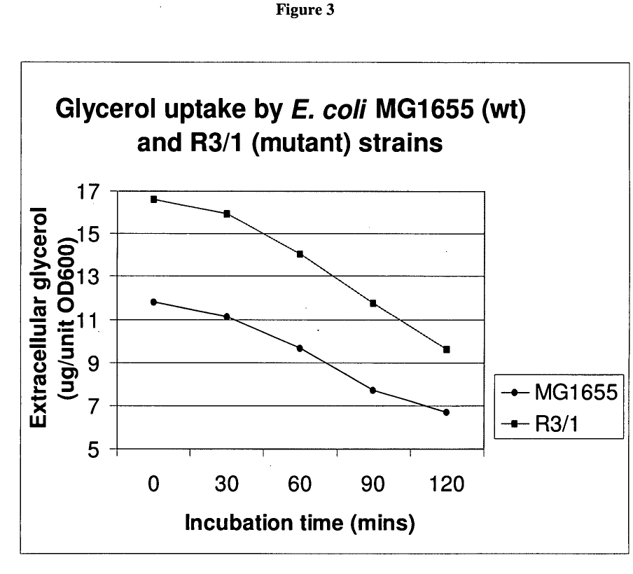

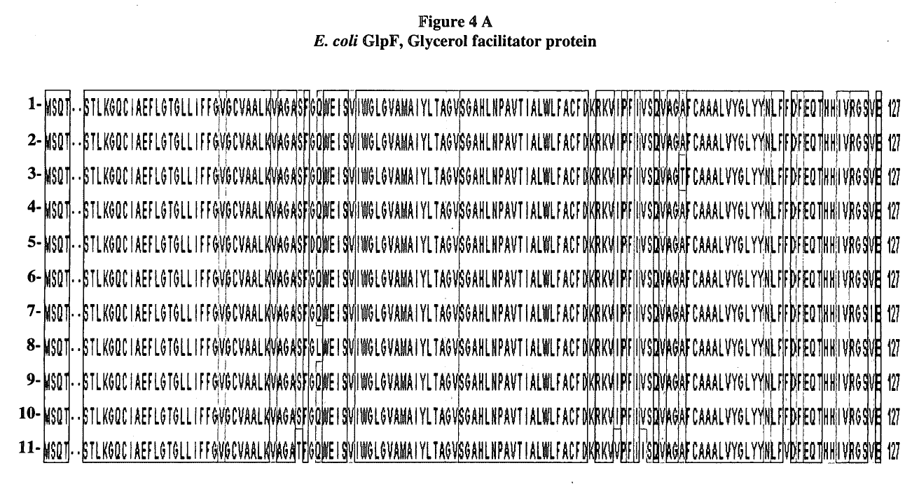

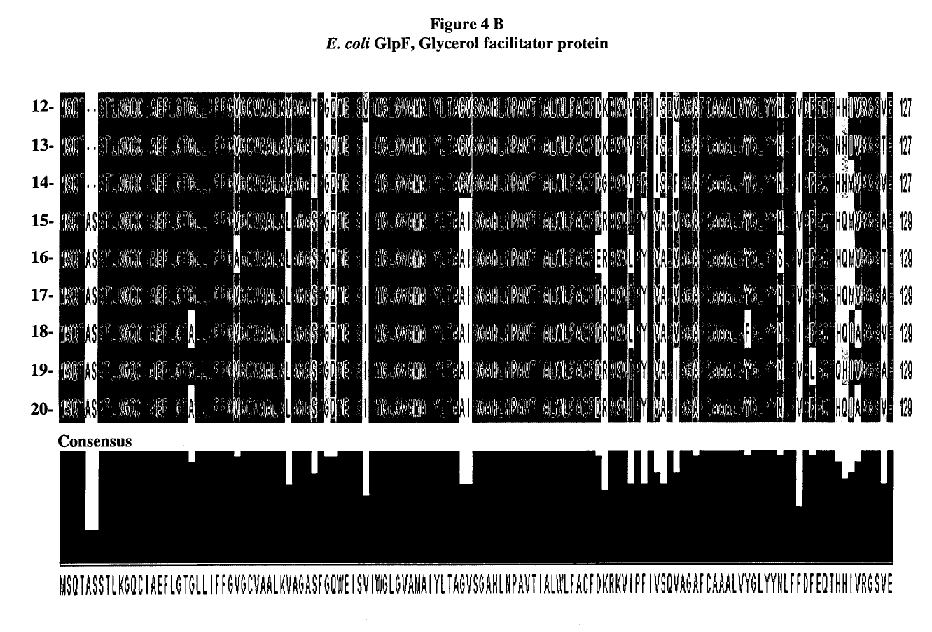

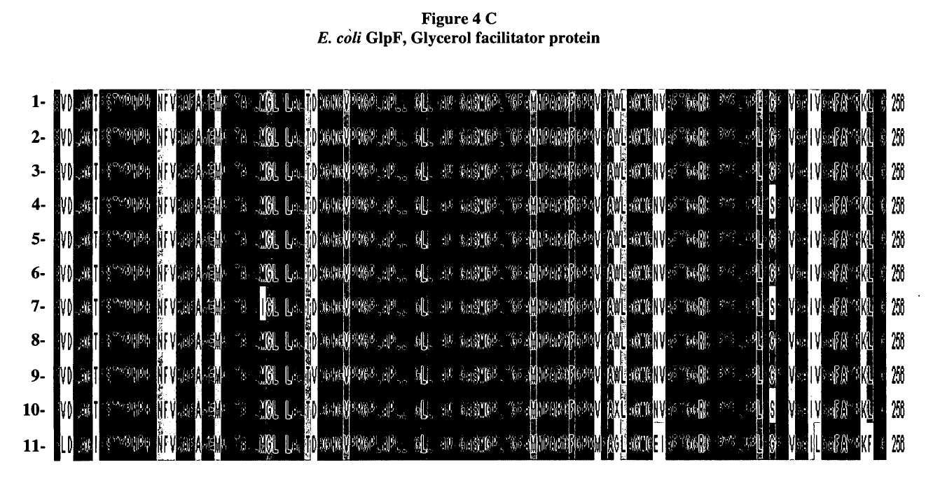

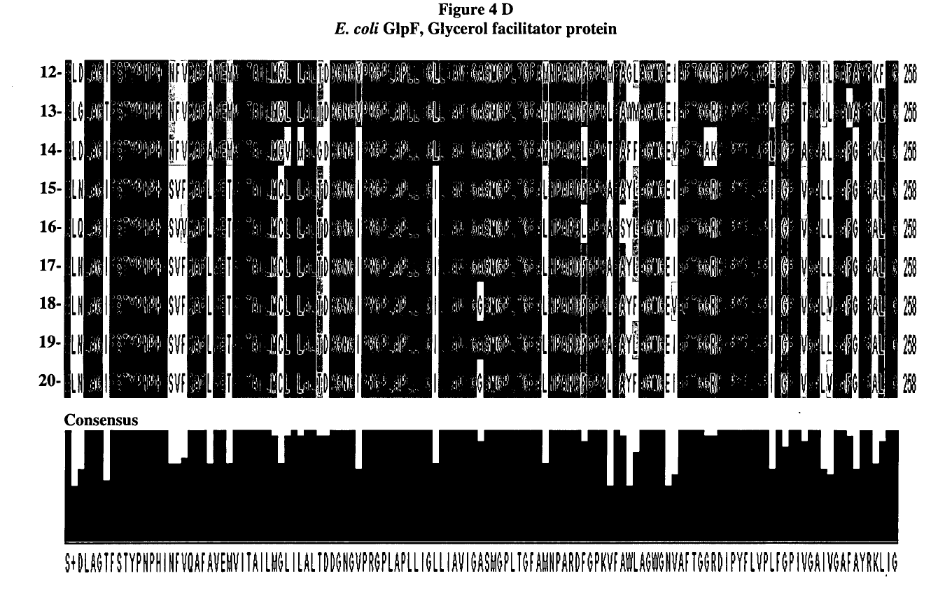

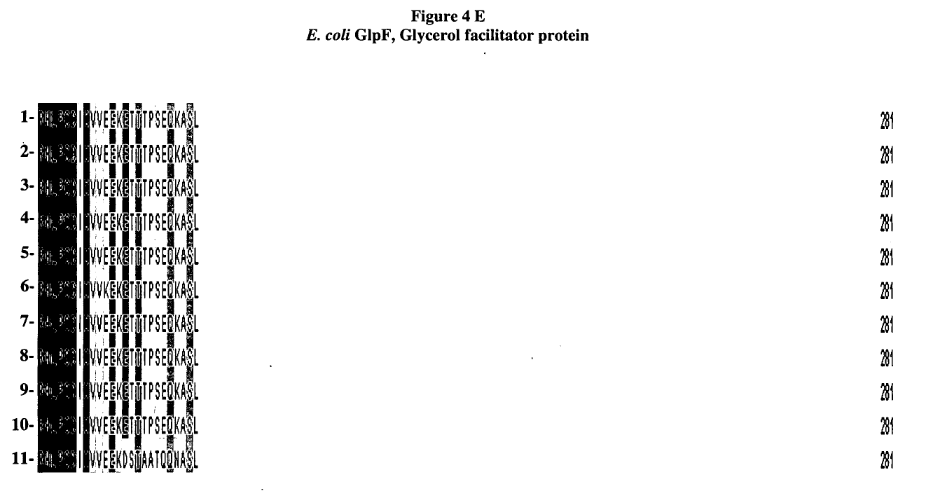

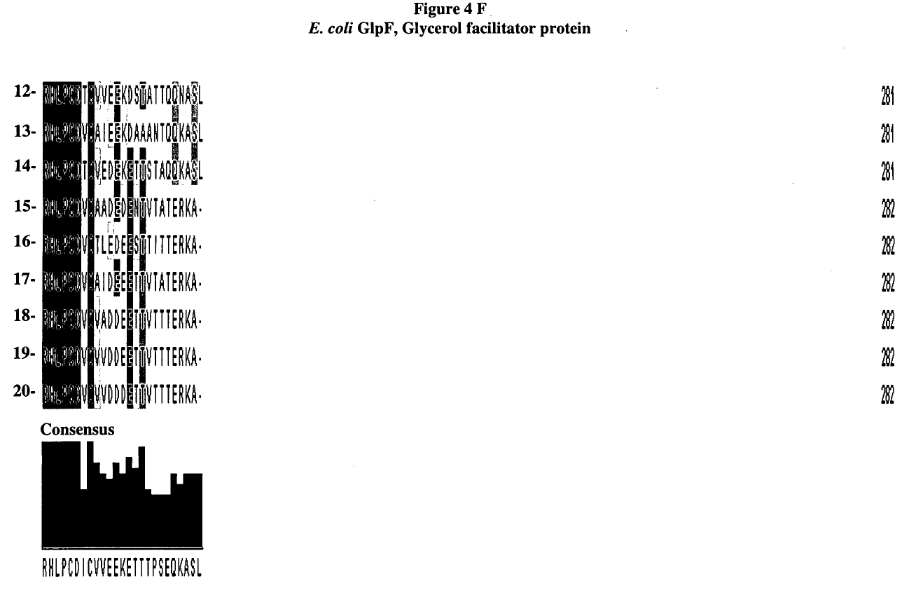

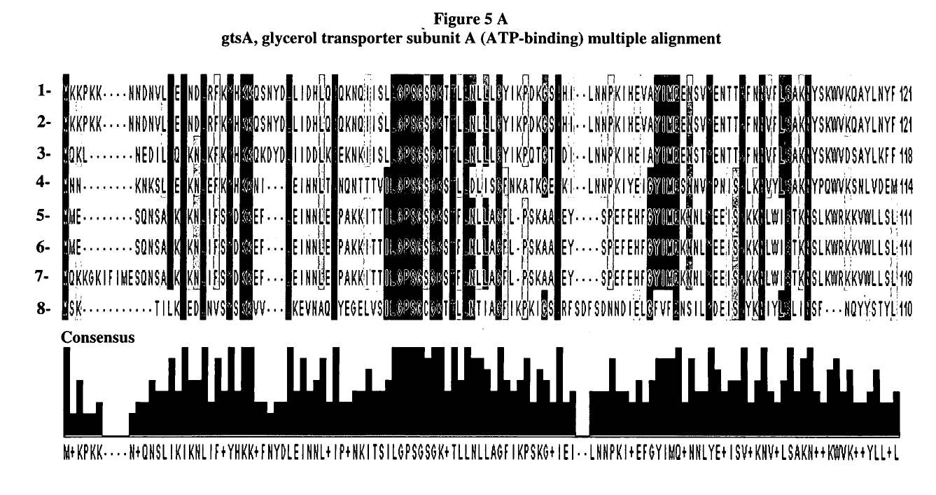

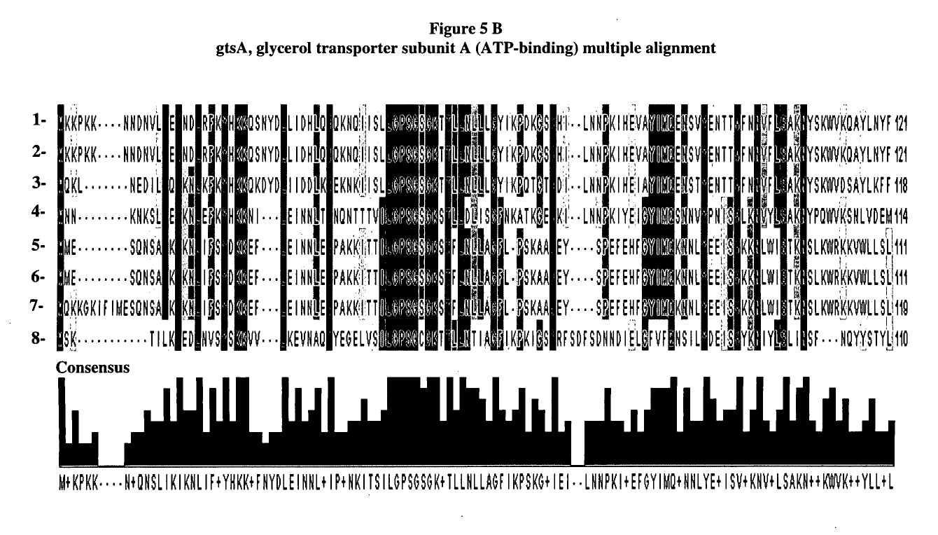

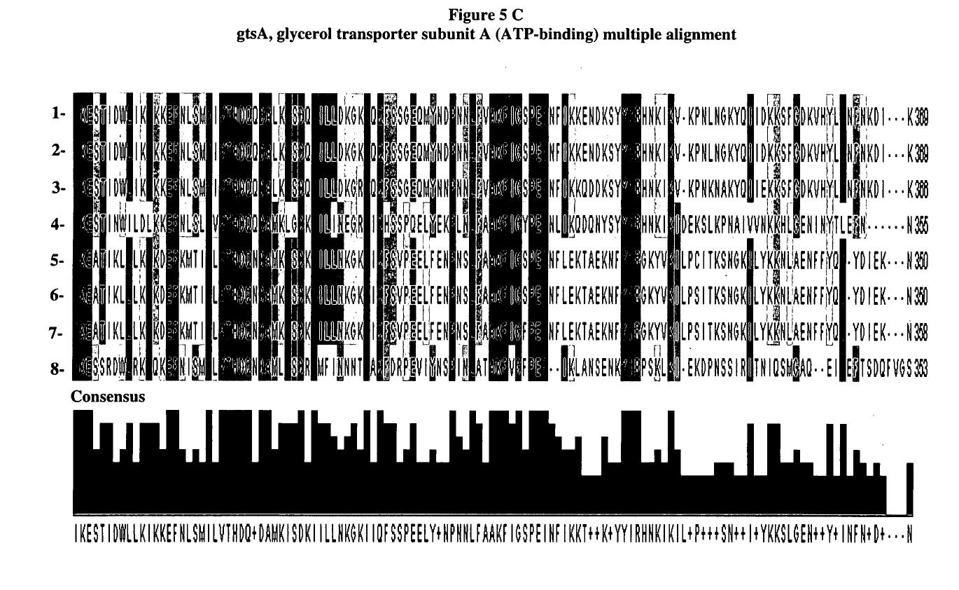

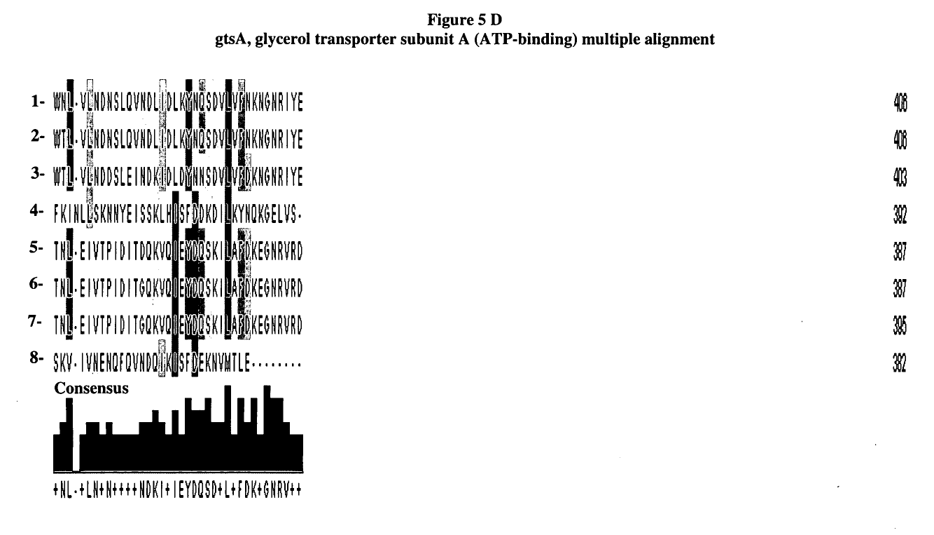

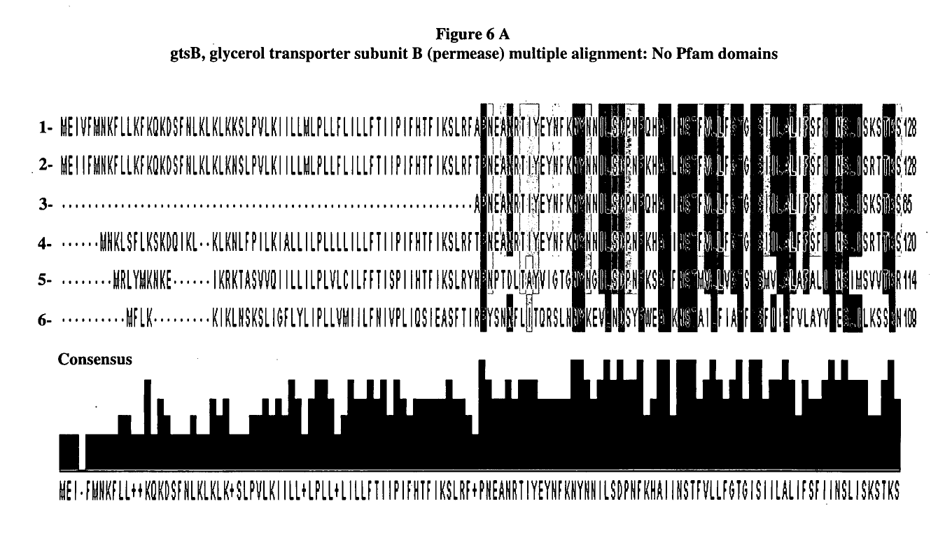

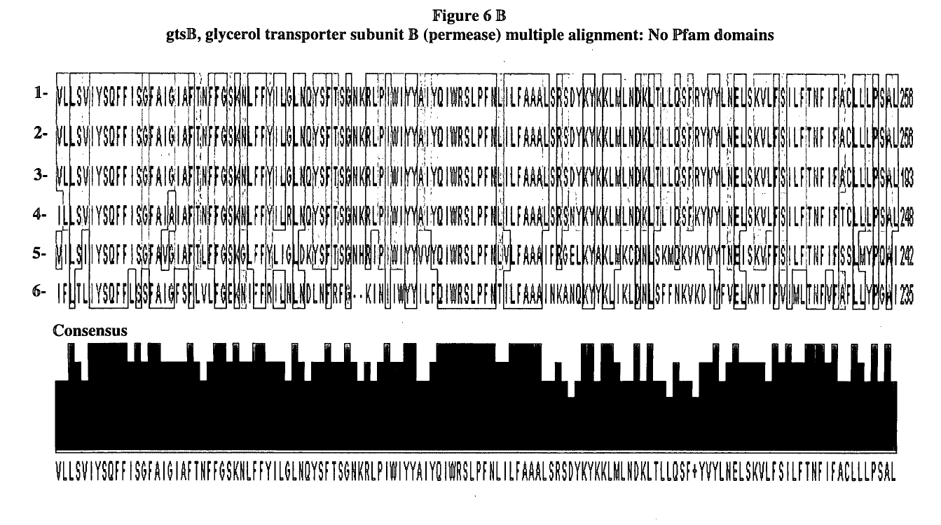

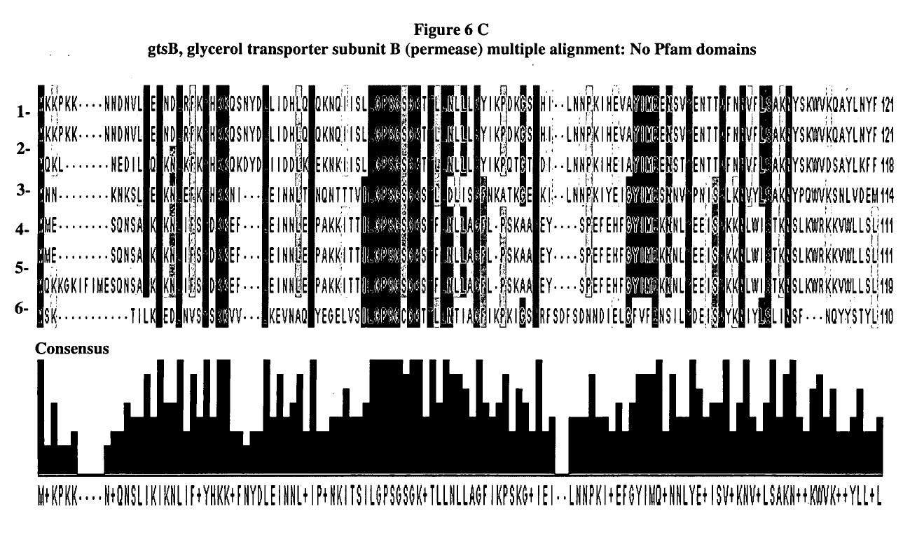

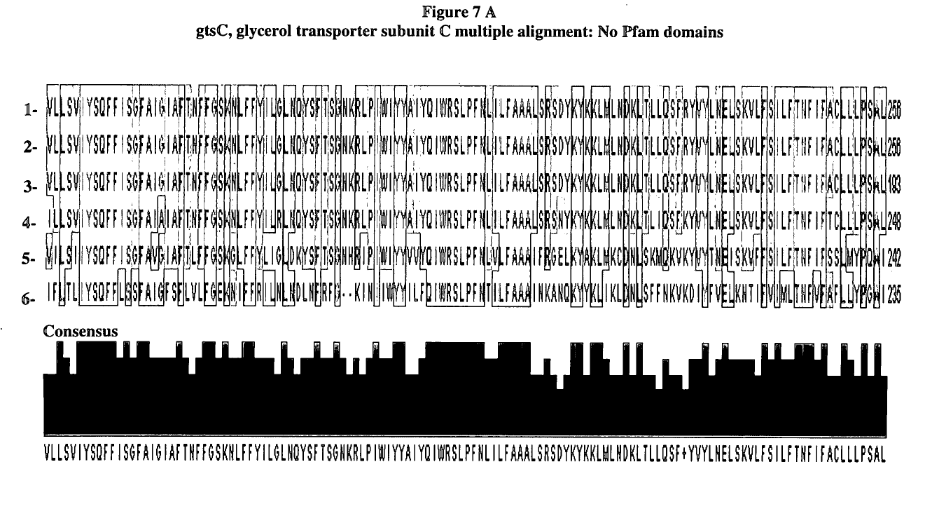

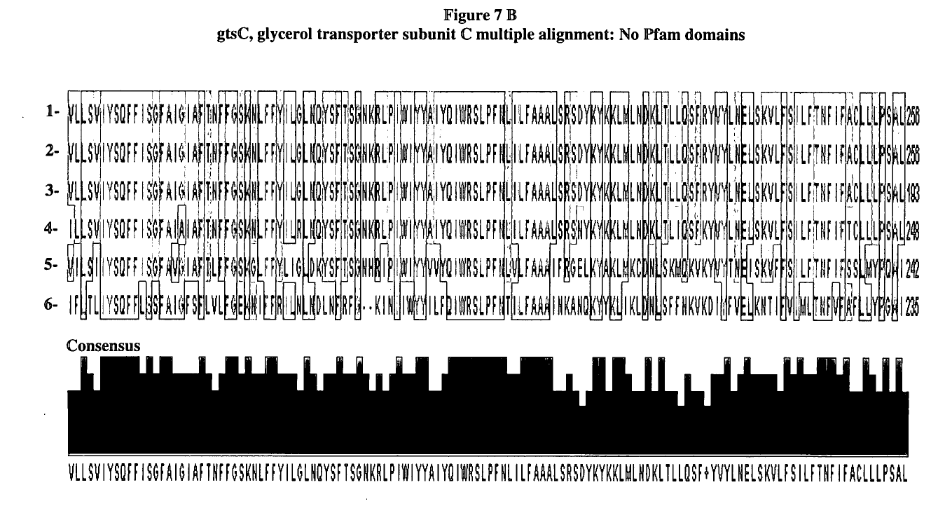

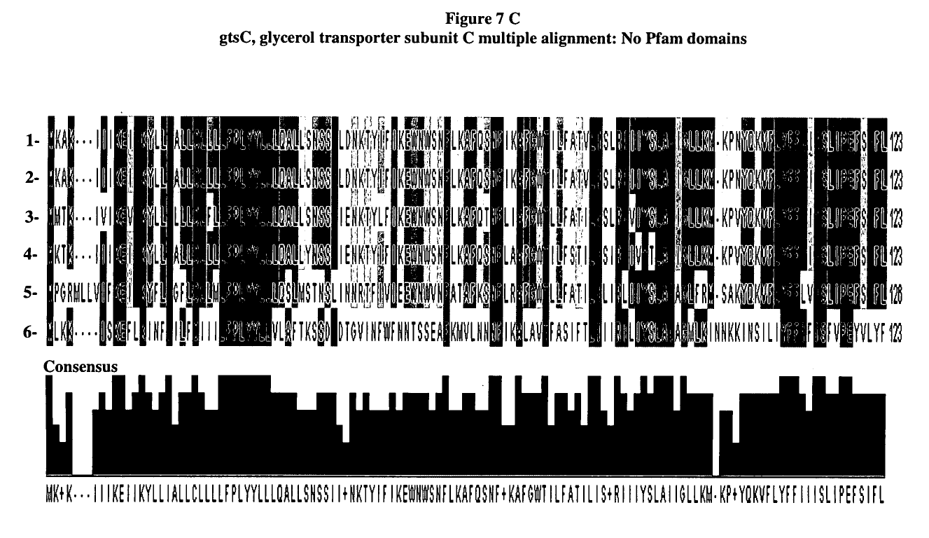

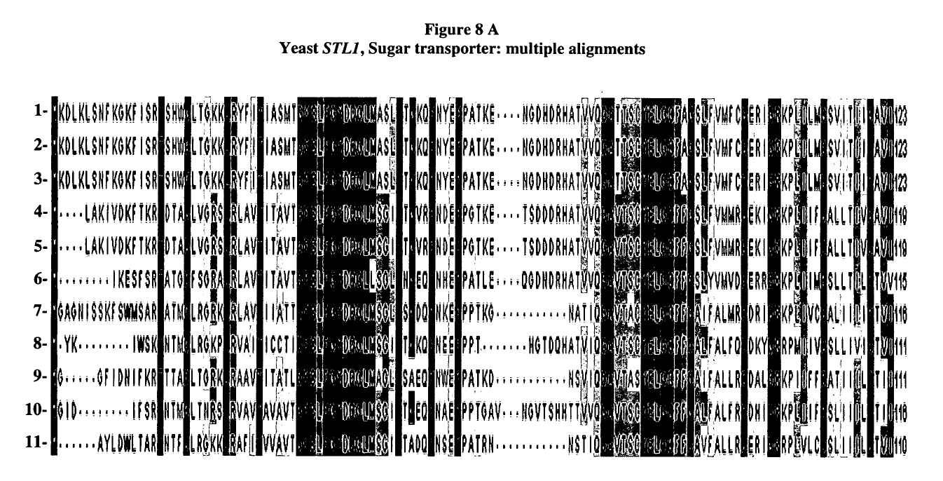

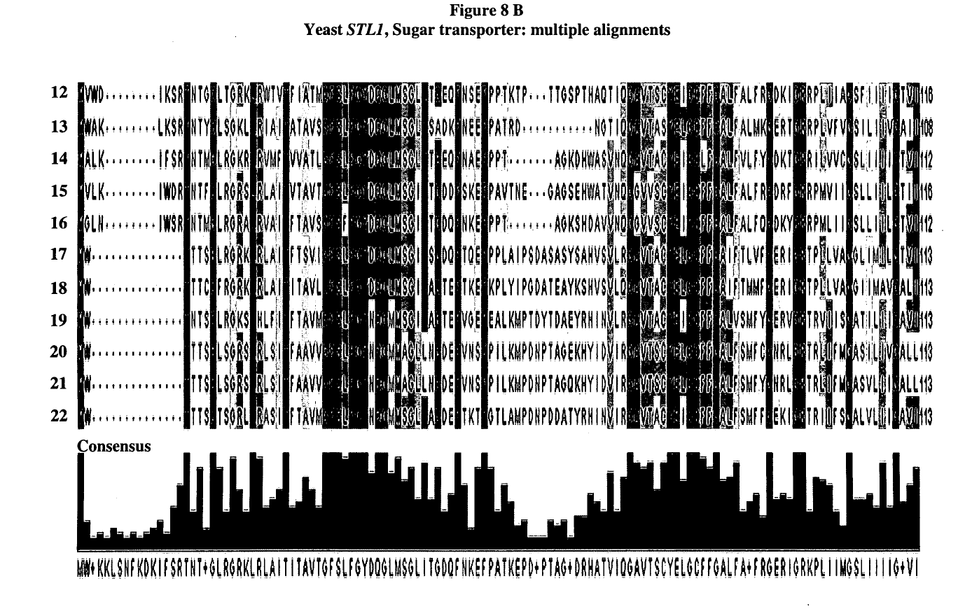

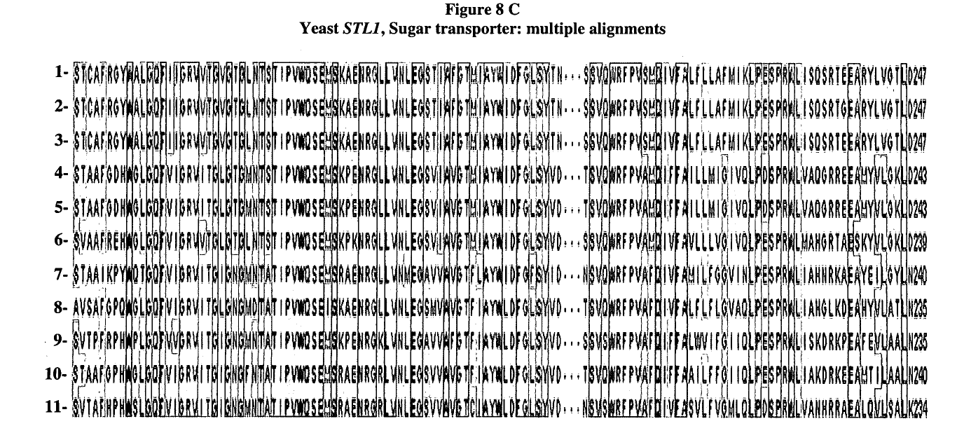

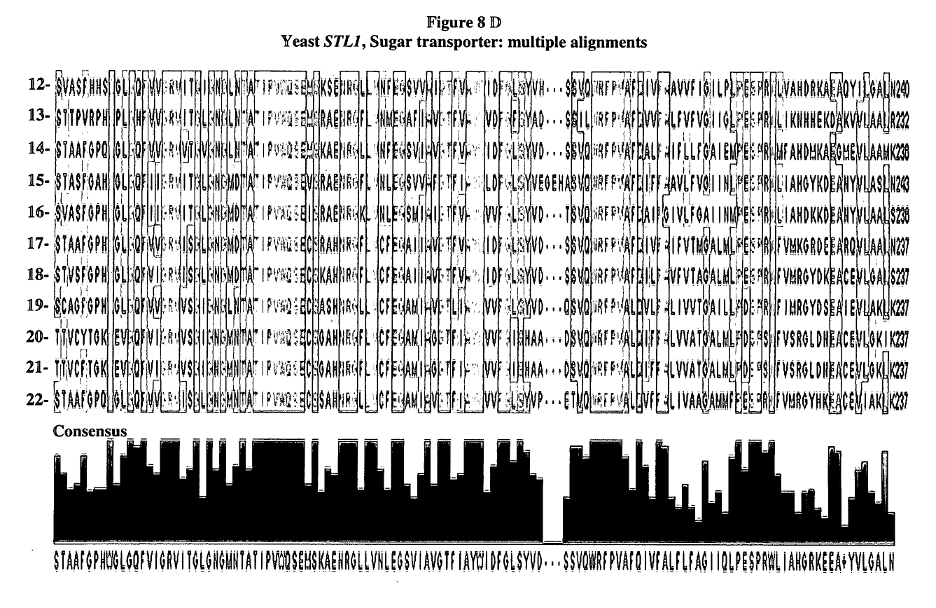

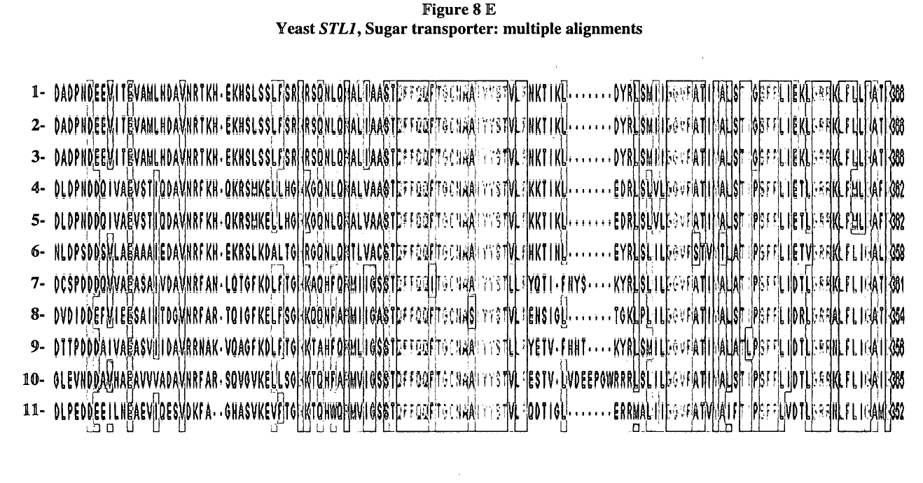

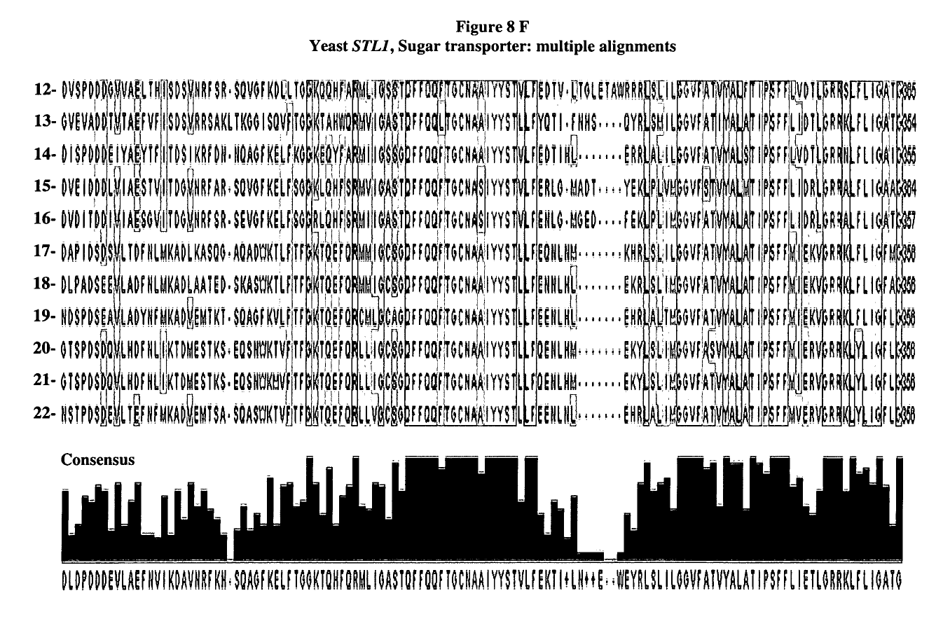

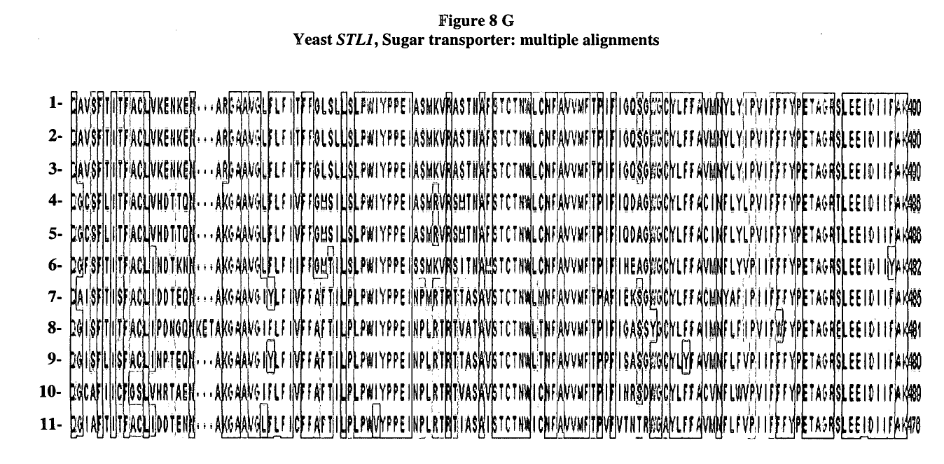

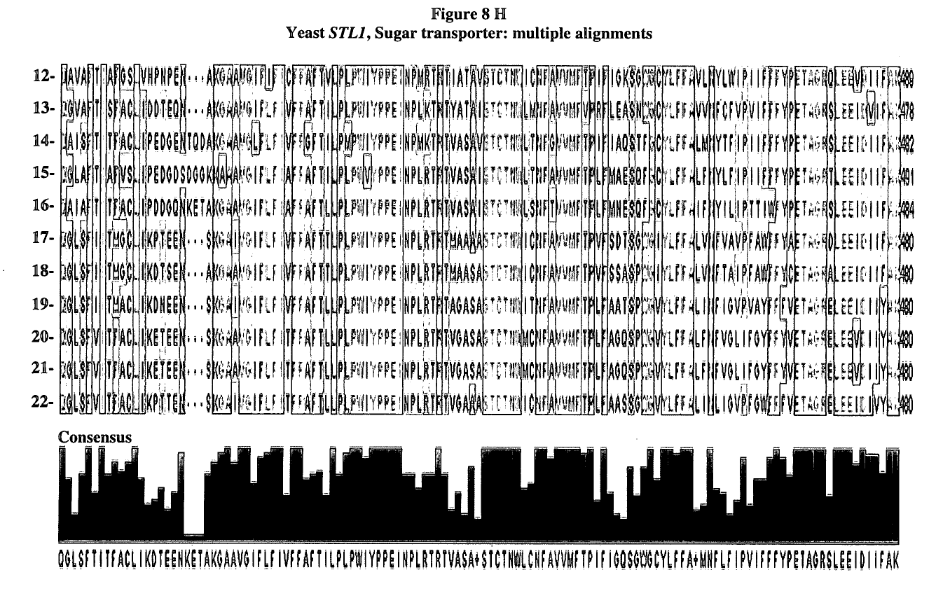

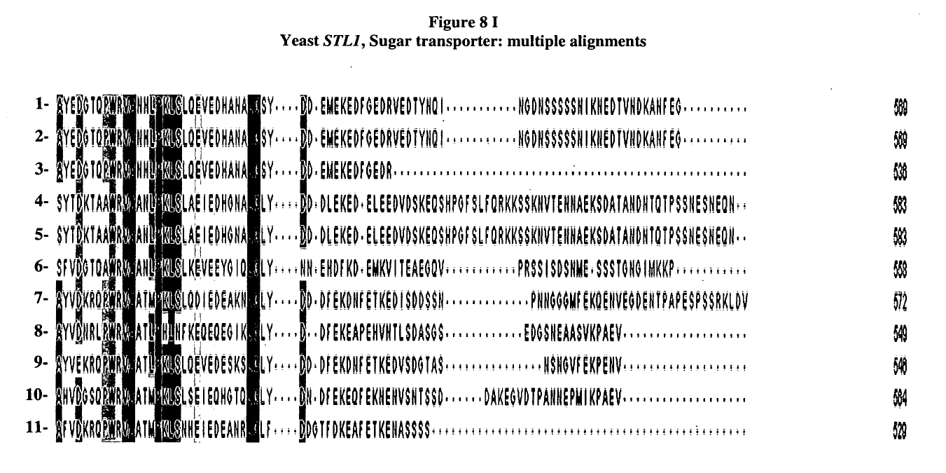

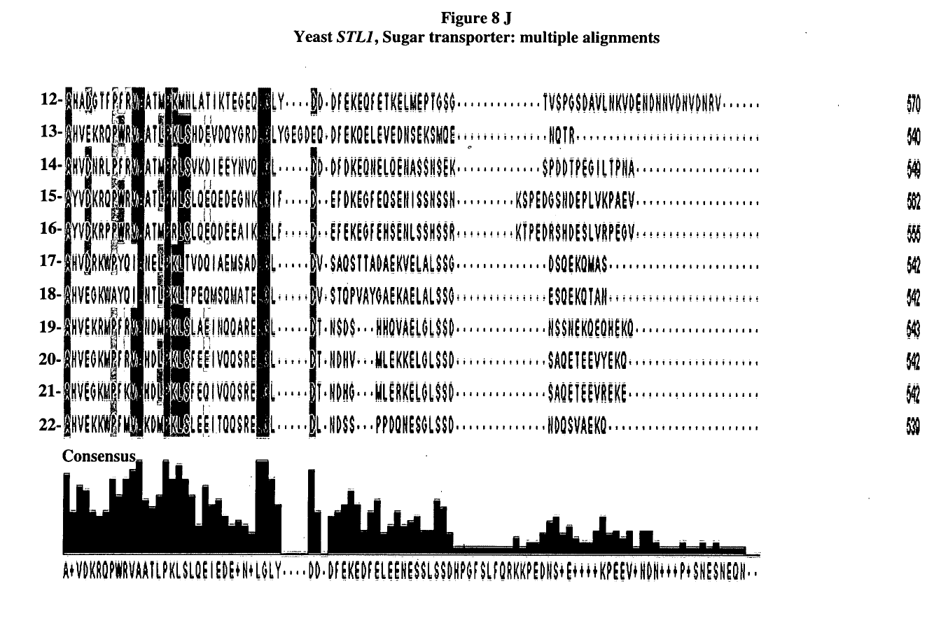

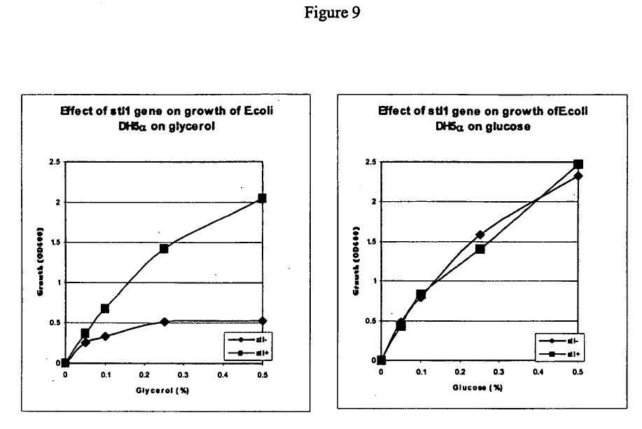

These experiments were performed in order to benchmark the E. coli R3/1 (ATCC as E. coli K12:MG1655-R3/1 as identifier IV638653-39710; ATCC No. PTA-8496)) mutant strain. The results demonstrate that there is no significant difference in the rate of glycerol utilization by wild type strain MG1655 and R3/1 mutant. See FIG. 3. Therefore, the mutation(s) in the R3/1 strain may confer upon the bacterium resistance to high glycerol concentrations without affecting the glycerol utilization pathway.

### Example 4

### Strain Growth in Glycerol-Containing Biodiesel Waste

In order to address whether bacteria such as E. coli, for instance, could grow on crude glycerol from biodiesel production-waste-streams from two commercial Chicago area biodiesel production facilities was obtained:

The biodiesel produced at site #1 was derived from soybean oil and other agricultural feedstocks. The biodiesel waste contained ˜92% glycerol.

The biodiesel generated from site #2 was derived from used cooking oil/fat employed by food service providers. The waste stream contained ˜90% glycerol.

The growth properties of wild-type E. coli strains MG1655 and W3100 together with the E. coli mutant R3 were tested to determine their relative ability to grow on glycerol-containing waste-streams from these biodiesel production sources. The results of this analysis are shown in Table 4 below. Table 4 shows the growth of E. coli strains on two different glycerol-containing biodiesel waste-streams. Numbers represent cell density (absorbance OD₆₀₀) values. E. coli MG1655 and W3100 are two different wild-type strains and R3 represents a glycerol-resistant mutant derived from E. coli MG1655.

TABLE 4

| | SITE #2 | | | | SITE #1 | | | |
|---|---|---|---|---|---|---|---|---|
| | 5% | | 10% | | 5% | | 10% | |
| BACTERIAL | GLYCEROL | | GLYCEROL | | GLYCEROL | | GLYCEROL | |
| STRAINS | 1 day | 2 day | 1 day | 2 day | 1 day | 2 day | 1 day | 2 day |
| MG1655 | 3.908 | 4.36 | 1.5 | 3.021 | 3.398 | 3.641 | 0.4272 | 0.767 |
| R3 | 3.832 | 4.4935 | 0.749 | 3.1725 | 3.0672 | 3.645 | 0.2468 | 0.2775 |
| mutant | | | | | | | | |
| W3100 | 3.978 | 3.4295 | 3.508 | 3.25 | 3.0368 | 2.8755 | 3.1206 | 3.1375 |

These studies indicate the E. coli (MG1655) R3 glycerol-resistant mutant strain shows slow but robust growth (in terms of cell density) at 5% glycerol that decreases slightly at 10% crude glycerol concentrations (Site #2) after 2 days. This growth decrease is possibly a consequence of the increased amounts of contaminating components in the waste-stream. Of particular importance is that the E. coli W3100 strain appears to display good growth properties in the 5% and 10% glycerol-containing biodiesel waste-streams from either production source. This suggested that this organism is a good host strain background from which to identify putative glycerol-resistant and/or glycerol tolerant mutants.

### Example 5

### STL1 Glycerol Active Transporter and Cell Growth

The proton/glycerol active transport protein encoded by the STL1 gene from the baker's yeast, S. cerevisiae was cloned into E. coli. The E. coli strain containing the heterologous STL1 gene increased bacterial growth (yield of biomass) on mineral medium with glycerol as the sole carbon source as compared with the host strain with no STL1 gene. See FIG. 9A. The STL1 gene did not affect bacterial growth on glucose used as a sole carbon source. Active proton-dependent glycerol transport into a bacterial cytoplasm may eliminate the limiting step in bacterial glycerol catabolism. In contrast, by way of a control, there is no effect of the presence or absence of STL1 on glucose utilization. See FIG. 9B.

Growth conditions: 0.1 ml of overnight culture grown on M9 mineral salt medium with glucose (0.5%), thiamine (1 mg/ml), casamino acids (0.005%) and kanamycin (50 μg/ml) was inoculated in flask containing 10 ml of the same medium and incubated with shaking (250 rpm) for 23 h at 37° C.

Strains: DH5α/TOPO II, DH5α/TOPO II/STL1. The STL1 gene was PCR cloned from the S. cerevisiae and cloned into pCRII-TOPO vector. The vector with the cloned STL1 gene was transformed into DH5a electro-competent cells. Genotype of parental DH5a is: F-Φ80dlac ZM15 (lacZYA-argE) U169 deoR recA1 endA1 hsdR17 (rk−, mk+) phoA supE44 thi-1 gyrA96 recA1.

Cloning of the yeast STL1 gene: The STL1 gene was PCR amplified from the S. cerevisiae and cloned into pCRII-TOPO vector (Stratagene, Calif.). The recombinant vector was transformed into E. coli DH5a electro-competent cells. Forward and reverse PCR primers (Sigma-Aldrich, St Louis, Mo.) to PCR amplify the S. cerevisiae STL1 gene comprised: 5′-TCAGCTTAGCTCAACCCTCAAAAT-3′ (SEQ ID NO:1) and 5′-TAAGGGAAACACTTTTGGTCCACC-3′ (SEQ ID NO:2). PCR reactions were performed using manufacturer's recommendations (Stratagene) using Easy-A High-Fidelity PCR Cloning Enzyme. PCR products were separated on a 0.7% agarose TBE gel, and the full-length amplified STL1 gene DNA fragment was excised and extracted from the gel by QIAquick Gel Extraction Kit (Qiagen, Calif.). Gel-purified STL1 DNA fragment was ligated into a pCRII-TOPO vector, and a proportion of the ligation mixture was transformed into DH5a electro-competent E. coli cells plated and selected for a selectable antibiotic marker (kanamycin). Transformants containing the cloned STL1 gene inserted into the pCRII-TOPO vector were validated using restriction enzyme digestion (using SpeI and XbaI) and nested PCR reactions. The correct validated clone of the STL1 gene in pCRII-TOPO vector in E. coli DH5a electro-competent cells was stored in a strain collection.

1. A glycerol utilizing composition comprising a microbial cell and a glycerol composition.

2. The microbial cell of claim 1, wherein the cell is characterized by increased resistance to glycerol toxicity.

3. The microbial cell of claim 1, wherein the cell is characterized by increased uptake of glycerol.

4. The microbial cell of claim 1, wherein the cell is characterized by increased glycerol metabolism.

5. The glycerol utilizing composition of claim 1, wherein the glycerol composition is crude glycerol.

6. The microbial cell of claim 2, wherein the increased resistance is derived from a nucleic acid isolated from a cell deposited in ATCC (identifier IV638653-39710; ATCC No. PTA-8496).

7. The cell deposited in the ATCC in claim 4.

8. The cell as in any one of the preceding claims, wherein the cell further comprises nucleic acid encoding a glycerol metabolizing system.

9. The cell as in any one of the preceding claims, wherein the cell further comprises a nucleic acid encoding a polypeptide selected from the group consisting glycerol facilitator, glycerol-specific energy-dependent transporter, and proton/glycerol symporter.

10. The cell of claim 8, wherein the nucleic acid encoding the glycerol metabolizing system is heterologous.

11. The cell of claim 10, wherein the proteins of the glycerol metabolizing system are selected from the group consisting of glycerol kinase, glycerol dehydrogenase, glycerol dehydratase, and 1,3-propanediol oxidoreductase.

12. The cell of claim 9, wherein the glycerol facilitator protein comprises GenBank Accession No. NP_{—}418362.1 or variants thereof.

13. The cell of claim 9, wherein the glycerol-specific energy-dependent transporter protein comprises GenBank Accession No. AF251037 or variants thereof.

14. The cell of claim 9, wherein the proton/glycerol symporter protein comprises GenBank Accession No. NP_{—}010825.1 or variants thereof.

15. A method for increasing glycerol utilization in a microbial cell comprising:
(a) providing a microbial cell selected from any one of the preceding claims; and
(b) contacting the microbial host cell with a composition comprising glycerol.

16. The method of claim 15, wherein the composition is a crude glycerol solution.

17. The method of claim 16, wherein the crude glycerol solution is obtained from the manufacture of biodiesel.

18. The method of claim 15, wherein step (b) is conducted under conditions suitable for the cell to produce a glycerol-derivable target compound.

19. The method of claim 15, wherein the target compound is selected from the group consisting of propionic acid, ethanol, 1,3-propanediol, 1,2-propanediol, 3-hydroxypropionic acid, poly (3-hydroxy-butyrate), poly (3-mercapto-propionate), hydrogen, succinate, dihydroxyacetone, butyric acid, acetic acid, polyglutamic acid, cinnamic acid, rhamnolipids, 3-hydroxacetone, omega-3 polyunsaturated fatty acids, and propylene glycol.


SEQUENCE LISTING (2 sequences)

SEQ ID NO: 1 - DNA, 24 bp, Artificial
  PCR primer for amplifying S. cerevisiae STL1 gene.
tcagcttagctcaaccctcaaaat

SEQ ID NO: 2 - DNA, 24 bp, Artificial
  PCR primer for amplifying S. cerevisiae STL1 gene.
taagggaaacacttttggtccacc